(19) United States
(12) Patent Application Publication
(10) Pub. No.: US 2024/0216510 A1
(43) Pub. Date: Jul. 4, 2024

(54) CHIMERIC ANTIGEN RECEPTORS TO SIALYL-TN GLYCAN ANTIGEN

(71) Applicants: RAMOT AT TEL-AVIV UNIVERSITY LTD., Tel Aviv (IL); ICHILOV TECH LTD., Tel Aviv (IL); YEDA RESEARCH & DEVELOPMENT CO. LTD., Rehovot (IL)

(72) Inventors: Vered PADLER-KARAVANI, Tel Aviv (IL); Ron AMON, Tel Aviv (IL); Lihi NINIOMANY, Tel Aviv (IL); Moran RAWET SLOBODKIN, Tel Aviv (IL); Anat GLOBERSON LEVIN, Tel Aviv (IL); Tova WAKS, Petach Tikva (IL); Zelig ESHHAR, Tel Itzhak (IL)

(21) Appl. No.: 18/556,172

(22) PCT Filed: Apr. 13, 2022
(86) PCT No.: PCT/IL2022/050384

§ 371 Date: Oct. 19, 2023

Related U.S. Application Data
(60) Provisional application No. 63/176,894, filed on Apr. 20, 2021.

(51) Int. Cl. A61K 39/00; A61P 35/00; C07K 14/705; C07K 14/725; C07K 16/30; C12N 5/0783

(52) U.S. Cl. CPC A61K 39/4631; A61K 39/4611; A61K 39/4613; A61K 39/464474; A61P 35/00; C07K 14/7051; C07K 14/70517; C07K 14/70521; C07K 14/70532; C07K 14/70575; C07K 14/70578; C07K 16/30; C12N 5/0636; A61K 2039/505; C07K 2317/622; C07K 2319/03; C07K 2319/30; C12N 2510/00

(57) ABSTRACT

The present invention discloses chimeric antigen receptors that specifically recognize and bind to Sialyl Tn carbohydrate antigen with high specificity and selectivity. The invention further provides lymphocytic cells, such as T cells, comprising said CARs, compositions comprising said cells or CARs as well as uses thereof.

### FIELD OF THE INVENTION

The present invention relates to chimeric antigen receptors (CARs) that specifically recognize and bind to Sialyl Tn carbohydrate antigen, cells expressing said CARs, compositions comprising said cells or CARs as well as uses thereof.

### BACKGROUND OF THE INVENTION

Chimeric antigen receptor (CAR) T cell therapy is one of the most growing fields in cancer therapy. The approval of CD19 directed CAR for treatment of acute lymphoblastic leukemia (ALL) and large B cell lymphoma lead to other trails to apply CD19 CAR for additional B cell malignancies. Moreover, efforts are made to develop novel CARs against tumor associated antigens against various cancers. Despite the great interest in novel CARS, therapy of solid tumors remains a considerable challenge. Administration of CAR against HER2 in human patients resulted in severe toxicity to lung tissue. Yet, additional attempts are made to utilize HER2 directed CARs in a different setup. Other novel targets for CARS for solid tumor therapy include IL13Ra2, epidermal growth factor receptor (EGFRvIII), carcino-embryonic antigen (CEA), Mesothelin and others. However, a major challenge in many of these targets is ‘on-target off-tumor’ toxicity. Low level expression of the CAR target in different tissues is sufficient to cause severe side effects and even fatal toxicity.

Tumor-associated carbohydrate antigens (TACA) are promising targets for many cancer types. Attempts are made to develop therapeutic molecules against these antigens in the form of monoclonal antibodies, bispecific antibodies, vaccines and CAR T. Sialyl-Tn (STn) is known for decades as tumor-associated carbohydrate antigen. STn levels were associated with tumor aggressiveness and resistant to chemotherapy. STn antigen is expressed in more than 80% of human carcinomas (Julien S et al., Biomolecules. 2012 Oct. 11; 2(4):435-66). U.S. Pat. No. 9,879,082 describes glycan-interacting antibodies and their use in the treatment and prevention of human diseases. U.S. Pat. No. 10,189,908 describes protein binding partners specific for glycopeptide variants associated with cancers. WO 2017/040529 discloses anti-sialyl Tn antigen (STn) CAR molecules and their use in treating, preventing, or ameliorating cancers that express STn expressing glycoproteins. Nevertheless, despite the abundance of different publications, there is no approved clinical therapeutics that target this antigen. There is an urgent need for development of efficient and safe therapies targeting STn and therefore treating a vast number of different tumors.

### SUMMARY OF THE INVENTION

According to one aspect, the present invention provides a chimeric antigen receptor (CAR) comprising an antigen binding domain that binds specifically to Sialyl Tn glycan (STn), wherein the antigen binding domain comprises three complementarity determining regions (CDRs) of a heavy-chain variable domain (VH) having amino acid sequence as set forth in SEQ ID NO: 1 and three CDRs of a light-chain variable domain (VL) having amino acid sequence as set forth in SEQ ID NO: 2. According to some embodiments, the VH-CDR1 comprises amino acid sequence SEQ ID NO: 3; VH-CDR2 comprises amino acid sequence SEQ ID NO: 4; VH-CDR3 comprises amino acid sequence SEQ ID NO: 5; VL-CDR1 comprises amino acid sequence SEQ ID NO: 6; VL-CDR2 comprises amino acid sequence SEQ ID NO: 7; and VL-CDR3 comprises amino acid sequence SEQ ID NO: 8. According to one embodiment, the VH domain comprises amino acid sequence SEQ ID NO: 1 or an analog thereof and the VL domain comprises amino acid sequence SEQ ID NO: 2 or an analog thereof, wherein the analog has at least 90% identity to said sequence. According to one embodiment, the VH domain consists of amino acid sequence SEQ ID NO: 1 or a functional analog thereof and the VL domain consists of amino acid sequence SEQ ID NO: 2 or a functional analog thereof, wherein the functional analog has at least 90% identity to said sequence. According to one embodiment, the wherein the VH and the VL domains are linked by a spacer to form a single chain variable fragment (scFv). According to one embodiment, the scFv comprises or consists of amino acid sequence SEQ ID NO: 11 or a functional analog thereof having at least 90% sequence identity to said sequence. According to any one of the above embodiments, the CAR comprises a transmembrane domain (TM domain), a costimulatory domain and an activation domain. According to some embodiments, the TM domain is a TM domain of a receptor selected from CD28 and CD8, or an analog thereof, and/or the costimulatory domain is selected from a costimulatory domain of a protein selected from CD28, 4-1BB, OX40, iCOS, CD27, CD80, and CD70, an analog thereof, and/or wherein the activation domain is selected from FcRγ and CD3-5 activation domains or an analog thereof, wherein the analog has at least 85% amino acid identity to the sequence. According to some embodiments, the CAR further comprising a leading peptide. According to some embodiments, the CAR of the present invention comprises amino acid sequence selected from SEQ ID NO: 15 and 16, and a functional analog thereof having at least 90% amino acid identity to said sequence. According to some embodiments, the CAR of the present invention consists of amino acid sequence selected from SEQ ID NO: 15 and 16, and a functional analog thereof having at least 90% amino acid identity to said sequence.

According to another aspect, the present invention provides a nucleic acid molecule encoding the CAR of the present invention. According to some embodiment, the nucleic acid molecule encodes amino acid sequence selected from SEQ ID NO: 1, SEQ ID NO: 2, both SEQ ID NOs: 1 and 2 and a functional analog thereof having at least 90% amino acid identity to said sequence. According to another embodiment, the nucleic acid comprises a nucleic acid sequence selected from SEQ ID NO: 17, SEQ ID NO: 18, a variant of SEQ ID NO: 17 or 18 having at least 95% sequence identity to the sequence(s), and a combination thereof. According to one embodiment, the nucleic acid of the present invention encodes amino acid sequence selected from SEQ ID NO: 11 and a functional analog thereof having at least 90% amino acid identity to said sequence. According to another embodiment, the nucleic acid comprises nucleic acid sequence selected from SEQ ID NO: 20 and a variant thereof having at least 95% sequence identity to said sequence. According to some embodiments, the nucleic acid further encoding an amino acid sequence selected from SEQ ID NOs: 12, 13, 14, an analog thereof, and any combination thereof, wherein the analog has at least 85% amino acid identity to said sequence. According to some embodiments, the nucleic acid of the present invention encodes amino acid sequence selected from SEQ ID NO: 15, SEQ ID NO: 16, and an analog thereof having at least 90% amino acid identity to said sequence. According to some embodiments, the nucleic acid of the present invention comprises a nucleic acid sequence selected from SEQ ID NO: 25, SEQ ID NO: 26, and a variant thereof having at least 90% sequence identity to the original sequence.

According to another aspect, the present invention provides a nucleic acid construct comprising the nucleic acid molecule of the present invention, operably linked to a promoter.

According to yet another aspect, the present invention provides a vector comprising the nucleic acid molecule or the nucleic acid construct of the present invention.

According to yet another aspect, the present invention provides a cell comprising the CAR, the nucleic acid molecule, the nucleic acid construct or the vector according to the present invention. According to some embodiments, the cell expresses or is capable of expressing the CAR of the present invention. According to some embodiments, the cell is selected from a T cell and a natural killer (NK) cell.

According to one aspect, the present invention provides a plurality of T cells comprising the CAR or the nucleic acid of the present invention.

According to another aspect, the present invention provides a pharmaceutical composition comprising a plurality of cells of the present invention, and a pharmaceutically acceptable carrier. According one embodiment, the cells are T cells. According one embodiment, the pharmaceutical composition comprising a plurality of T cells comprising the CAR of the present invention, and a pharmaceutically acceptable carrier. According to some embodiments, the pharmaceutical composition of the present invention is for use in treating cancer. According to one embodiment, the cancer is selected from a carcinoma and lymphoma. According to one embodiment, the cancer is selected from endometrial carcinoma, breast cancer, ovarian carcinoma, prostate adenocarcinoma, seminoma, diffuse type gastric adenocarcinoma, pancreatic and colon adenocarcinomas, lung adenocarcinoma and mantle cell lymphoma.

According to a further aspect, the present invention provides a method for treating cancer in a subject in need thereof comprising administering a therapeutically effective amount of T cells or the pharmaceutical composition of the present invention.

### BRIEF DESCRIPTION OF DRAWINGS

FIG. 1 shows the specificity of the full-length chimeric antibody RA0 anti-STn hIgG (0.2 ng/μl) as examined by ELISA inhibition assay against coated STn-PAA-Biotin, after pre-incubation of the antibody with specific (STn; Neu5Acα2-6GalNAcα) or non-specific glycans (Neu5Acα2-3GalNAcα, Tn and SLe^{a}) [0.3-300 nM; all glycans conjugated to biotinylated polyacrylamide (PAA-Bio); mean±STD].

FIG. 2 shows the summary of immunohistochemistry staining of human cancers tissue microarray slides containing twenty-three different cancer tissues using biotinylated RA0 antibody (Bio-RA0-hIgG; 10 ng/μl), followed by incubation HRP-streptavidin and DAB staining. This staining indicates the presence of STn in endometrial carcinoma, ovarian carcinoma, prostate adenocarcinoma, seminoma, diffuse type gastric adenocarcinoma, pancreatic and colon adenocarcinomas, lung adenocarcinoma and mantle cell lymphoma (Bar 100 μm; representative of two independent experiments).

FIG. 3 shows the binding of RA0-hIgG antibody to B16F10 mouse melanoma cancer cell line and their cytotoxicity. FIG. 3A—B16F10 mouse melanoma cell line was stained with RA0-IgG (10 ng/μl) then detected with Cy3-goat-anti-human IgG (H+L) (1:100) then read by FACS. Secondary antibody only was used as a negative control, confirming STn expression on B16F10 target cells. FIG. 3B—RA0-hIgG show dose-dependent complement-dependent cytotoxicity (CDC) against B16F10 target cells (RA0 at 4 ng/μl and 2 ng/μl), as determined by LDH detection kit. (Mean±SEM; representative of two independent experiments).

FIG. 4 shows a schematic presentation of the constructed CAR comprising RA0 scFv.

FIG. 5 shows RA0-CAR specificity against the tumor-associated carbohydrate antigen STn. FIG. 5A shows the specificity of RA0-CAR expressing T cells against the STn target antigen that was evaluated by FACS, in comparison to the closely-related non-specific Tn glycan target. As a negative control, irrelevant-N29-CAR expressing T cells (targeting ErbB2) were used. Transduced CAR T cells were incubated with biotinylated-polyacrylamide-conjugated glycan antigens (glycan-PAA-Bio at 1 μM each), then binding detected with APC-streptavidin (APC-SA) (representative of two independent experiments). FIG. 5B—Geometric mean of glycan binding by RA0-CAR T cells (APC detection) of FACS staining from (FIG. 5A).

FIG. 6 shows in vitro stimulation of RA0-CAR T cells with properly presented glycan antigen. RA0-CAR T cells or control untransduced T cells (UT) were added to wells coated with cither monovalent STn-Bio or polyvalent STn-PAA-Bio (FIG. 6A, left and right structures, respectively) for overnight incubation at 37° C. FIGS. 6B and 6C show the levels of secreted IFN-γ and TNF-α, respectively, in the growth media as measured by ELISA (mean±STD). FIG. 6D shows secretion of IFN-γ levels in the growth media after co-culturing of RA0-CAR T cells or control (untransduced T cells (UT)) with Raji, Capan-2, MEG-01 and FaDu cells at 2:1 E:T ratio.

FIG. 7 shows in vivo cytotoxicity of RA0-CAR T cells against B16F10 cancer cells in mice. C57BL/6 mice were injected subcutaneously with 0.25×10⁶ B16F10 cells. In the first in vivo experiment (treatment regimen 1; FIG. 7A-C), on day 10 mice were irradiated at 2 Gy, and on the following day treated by intravenous (systemic) injections of 7×10⁶ RA0-CAR T cells (˜50% transduction) or control untransduced T cells (UT) (n=5 per group). In a second in vivo experiment (treatment regimen 2; FIG. 7D) on day 3, mice were irradiated at 2 Gy, and on the following day treated by intravenous (systemic) injections of 7×10⁶ RA0-CAR T cells (˜50% transduction) or control untransduced T cells (UT) (n=5 per group). Tumor volume was monitored every other day. In treatment regimen 1, tumor volume measurements per mouse was higher in control (UT) treated mice (FIG. 7A) compared to the RA0-CAR treated micc (FIG. 7B). FIG. 7C shows that in treatment regimen 1, the mean tumor volume of the RA0-CAR T treated group was significantly lower than that of the control UT cells treated group (Mean±SEM; Two-way ANOVA with Sidak post-tests, * p<0.05, *** p<0.0001). FIG. 7D shows that in treatment regimen 2, the mean tumor volume of the RA0-CAR T treated group was significantly lower than that of the control UT cells treated group (Mean±SEM; Two-way ANOVA with Sidak post-tests, *** p<0.001). Together all data show that adoptive transfer of RA0-CAR T cells inhibited growth of STn-expressing B16F10 melanoma tumors compared to the UT controls.

### DETAILED DESCRIPTION OF THE INVENTION

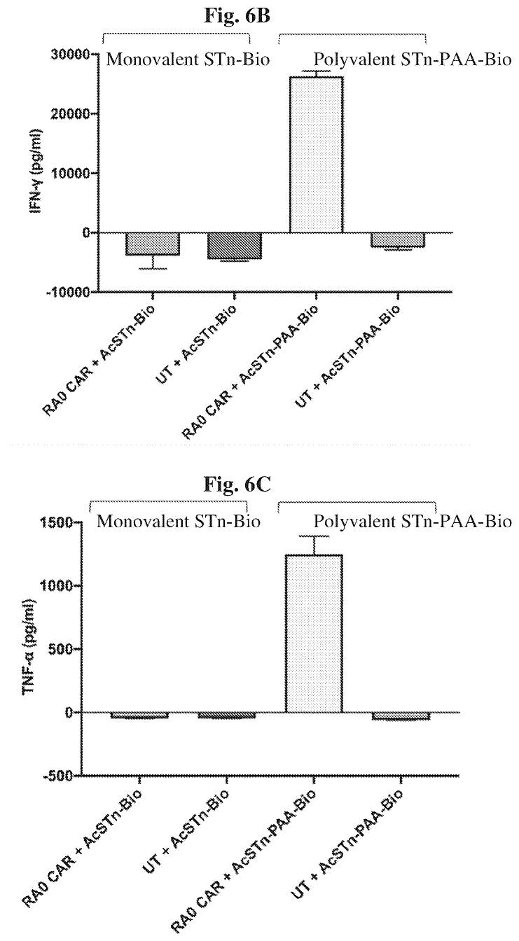

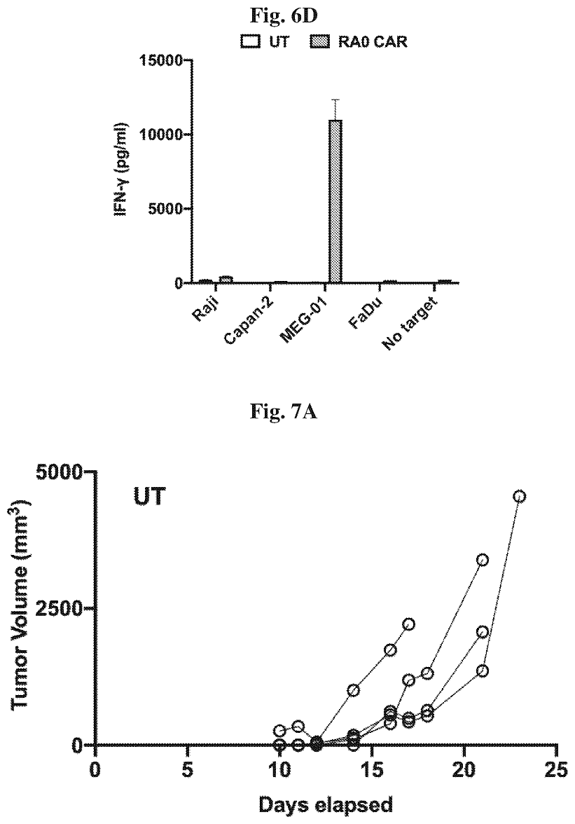

The present invention discloses chimeric antigen receptors comprising an antigen binding domain (ABD) of RA0 antibody that specifically bind to Sialyl Tn glycan. The invention is based, inter alia, on data showing that chimeric antigen receptors (CARs) comprising an antigen binding domain (ABD) of RA0 antibody that specifically bind to Sialyl Tn glycan, upon binding to STn glycan successfully stimulates the engineered T-cells presenting said CAR, and that these T-cells induce cytotoxicity towards cancer cells presenting STn (as shown in FIG. 6C). Moreover, as shown in Example 2, stimulation of CAR T-cells specific to STn was observed only when they were incubated with cancer cells presenting said antigen but not with normal cells. This indicates that treatment with such CAR T-cells is highly specific to cell expressing STn glycan, safe and does not induce off-target cytotoxicity.

According to one aspect, the present invention provides a chimeric antigen receptor (CAR) comprising an antigen binding domain that binds specifically to Sialyl Tn glycan (STn), wherein the antigen binding domain comprises three complementarity determining regions (CDRs) of a heavy-chain variable domain (VH) having amino acid sequence as set forth in SEQ ID NO: 1 and three CDRs of a light-chain variable domain (VL) having amino acid sequence as set forth in SEQ ID NO: 2.

The terms “chimeric antigen receptor” or “CAR” are used herein interchangeably and refer to engineered recombinant polypeptide or receptor which is grafted onto cells and comprises at least (1) an extracellular domain comprising an antigen-binding region, e.g., a single chain variable fragment of an antibody or a whole antibody, (2) a transmembrane domain to anchor the CAR into a cell, and (3) one or more cytoplasmic signaling domains (also referred to herein as “an intracellular signaling domains”). The extracellular domain comprises an antigen binding domain (ABD) and optionally a spacer or hinge region. The antigen binding domain of the CAR targets a specific antigen. The targeting regions may comprise full length heavy chain, Fab fragments, or single chain variable fragment (scFv).

The terms “antigen binding portion”,” antigen binding domain” and “ABD” as used herein refer to one or more fragments of an antibody that retain the ability to specifically bind to an antigen. It has been shown that the antigen binding function of an antibody can be performed by fragments of a full-length antibody. Such ABD may also be bispecific, dual specific, or multi-specific formats; specifically binding to two or more different antigens. Examples of binding fragments encompassed within the term “antigen binding portion” include (i) a Fab fragment, a monovalent fragment consisting of the VL, VH, CL and CH1 domains; (ii) a F(ab′)2 fragment, a bivalent fragment comprising two Fab fragments linked by a disulfide bridge at the hinge region; (iii) a Fd fragment consisting of the VH and CH1 domains; (iv) a Fv fragment consisting of the VL and VH domains of a single arm of an antibody, (v) a dAb, which comprises a single variable domain; and (vi) an isolated complementarity determining region (CDR). Furthermore, although the two domains of the Fv fragment, VL and VH, are coded for by separate genes, they can be joined, using recombinant methods, by a synthetic linker that enables them to be made as a single protein chain in which the VL and VH regions pair to form monovalent molecules known as single chain Fv (scFv). Such single chain antibodies are also intended to be encompassed within the term “antigen binding portion”. In certain embodiments of the invention, scFv molecules are incorporated into a fusion protein. Other forms of single chain antibodies, such as diabodies are also encompassed. The antigen binding domain can be derived from the same species or a different species for or in which the CAR will be used. In one embodiment, the antigen binding domain is a scFv.

The terms “light chain variable region”, “VL” and “VI.” are used herein interchangeably and refer to a light chain variable region of an antibody capable of binding to STn glycan. The terms “heavy chain variable region”, “VH” and “VH” are used herein interchangeably and refer to a heavy chain variable region of an antibody capable of binding to STn glycan.

According to any one of the above embodiments, the VL and VH domains may be linked by to form a single chain variable fragment (scFv). VL and VH domains in the scFv may be in any order, such as N′-VH-VL-C′ or N′-VL-VH-C′. The VH and VL domains may be linked by a linker.

According to some embodiments, the extracellular domain comprises a hinge region. The extracellular spacer or hinge region of a CAR is located between the antigen binding domain and a transmembrane domain. Extracellular spacer domains may include, but are not limited to, Fc fragments of antibodies or fragments or derivatives thereof, hinge regions of antibodies or fragments or derivatives thereof, constant domains such as CH2 region or CH3 region of antibodies, accessory proteins, artificial spacer sequences or combinations thereof.

As used herein, the term “CDR” refers to the complementarity determining region within antibody variable sequences. There are three CDRs in each of the variable regions of the heavy chain and the light chain, which are designated CDR1, CDR2 and CDR3 (or specifically VH CDR1, VH CDR2, VH CDR3, VL CDR1, VL CDR2, and VL CDR3), for each of the variable regions. The exact boundaries of these CDRs have been defined differently according to different systems. The system described by Kabat (Kabat et al., Sequences of Proteins of Immunological Interest (National Institutes of Health, Bethesda, Md. (1987) and (1991)) not only provides an unambiguous residue numbering system applicable to any variable region of an antibody, but also provides precise residue boundaries defining the three CDRs. Still other CDR boundary definitions may not strictly follow one of known systems, but will nonetheless overlap with the Kabat CDRs, although they may be shortened or lengthened in light of prediction or experimental findings that particular residues or groups of residues or even entire CDRs do not significantly impact antigen binding. Determination of CDR sequences from antibody heavy and light chain variable regions can be made according to any method known in the art, including but not limited to the methods known as KABAT, Chothia and IMGT. The selected set of CDRs may include sequences identified by more than one method, namely, some CDR sequences may be determined using KABAT and some using IMGT. According to one embodiment, the CDRs are defined using KABAT method.

As used herein, the terms “framework”, “framework domain”, “framework region” or “framework sequence” refer to the remaining sequences of a variable region minus the CDRs. Because the exact definition of a CDR sequence can be determined by different systems, the meaning of a framework sequence is subject to correspondingly different interpretations. The six CDRs also divide the framework regions on the light chain and the heavy chain into four sub-regions (FR1, FR2, FR3 and FR4) on each chain, in which CDR1 is positioned between FR1 and FR2, CDR2 between FR2 and FR3, and CDR3 between FR3 and FR4. Without specifying the particular sub-regions as FR1, FR2, FR3 or FR4, a framework region, as referred by others, represents the combined FR's within the variable region of a single, naturally occurring immunoglobulin chain. As used herein, a FR represents one of the four sub-regions, and FRs represent two or more of the four sub-regions constituting a framework region.

The term “transmembrane domain” refers to the region of the CAR, which crosses or bridges the plasma membrane. The transmembrane domain of the CAR of the invention is the transmembrane region of a transmembrane protein, an artificial hydrophobic sequence or a combination thereof. According to some embodiments, the term comprises also transmembrane domain together with extracellular spacer or hinge region.

The term “intracellular domain” refers to the intracellular part of the CAR and may be an intracellular domain of T cell receptor or of any other receptor (e.g., TNFR superfamily member) or portion thereof, such as an intracellular activation domain (e.g., an immunoreceptor tyrosine-based activation motif (ITAM)-containing T cell activating motif), an intracellular costimulatory domain, or both.

The terms “binds specifically” or “specific for” with respect to an antigen-binding domain of an antibody or of a fragment thereof refers to an antigen-binding domain which recognizes and binds to a specific antigen, but does not substantially recognize or bind other molecules in a sample. The term contemplates that the antigen-binding domain binds to its antigen with high affinity and binds other antigens with low affinity. An antigen-binding domain that binds specifically to an antigen from one species may bind also to that antigen from another species. This cross-species reactivity is not contrary to the definition of that antigen-binding domain as specific. The term “KD”, as used herein, is intended to refer to the dissociation constant of a particular antibody-antigen interaction. Kp is calculated as kₐ/k_{d}. The term “kₒₙ” or “kₐ”, as used herein, is intended to refer to the on-rate constant for association of an antibody to the antigen to form the antibody/antigen complex. The term “K_{off}” or “k_{d}”, as used herein, is intended to refer to the off-rate constant for dissociation of an antibody from the antibody/antigen complex.

The terms “Sialyl Tn glycan”, “Sialyl Tn” and “STn” are used herein interchangeably and refer Neu5Acα2-6GalNAcαO(CH2)₂CH₂NH₂ disaccharide carbohydrate having the structure as presented in Structure I.

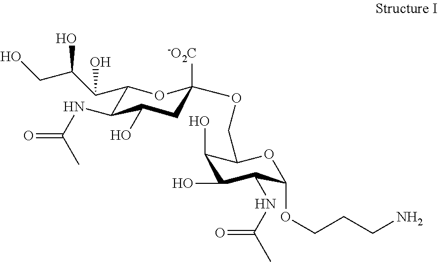

According to some embodiments, the CAR comprises an antigen binding domain that binds specifically to Sialyl Tn glycan (STn), wherein the antigen binding domain comprises three VH-CDRs and three VL-CDR, wherein the VH-CDR1 comprises amino acid sequence SEQ ID NO: 3; VH-CDR2 comprises amino acid sequence SEQ ID NO: 4; VH-CDR3 comprises amino acid sequence SEQ ID NO: 5; VL-CDR1 comprises amino acid sequence SEQ ID NO: 6; VL-CDR2 comprises amino acid sequence SEQ ID NO: 7; and VL-CDR3 comprises amino acid sequence SEQ ID NO: 8. According to some embodiments, the CAR of the present invention comprises an antigen binding domain that binds specifically to Sialyl Tn glycan (STn), wherein the antigen binding domain comprises three VH-CDRs and three VL-CDR, wherein the VH-CDR1 consists of the amino acid sequence SEQ ID NO: 3; VH-CDR2 consists of the amino acid sequence SEQ ID NO: 4; VH-CDR3 consists of the amino acid sequence SEQ ID NO: 5; VL-CDR1 consists of the amino acid sequence SEQ ID NO: 6; VL-CDR2 consists of the amino acid sequence SEQ ID NO: 7; and VL-CDR3 consists of the amino acid sequence SEQ ID NO: 8.

According to some embodiments, the ABD comprises a VH domain comprising amino acid sequence SEQ ID NO: 1 or an analog thereof and a VL domain comprises amino acid sequence SEQ ID NO: 2 or an analog thereof, wherein the analog has at least 90% sequence identity to said sequence.

The term “analog” refers to a polypeptide, peptide or protein which differs by one or more amino acid alterations/modification (e.g., substitutions, additions or deletions of amino acid residues) from the original sequence, having at least 85% sequence identity to the original sequence and still maintains the properties of the parent polypeptide, peptide or protein. According to one embodiment, the analog comprises at least one modification selected from a substitution, deletion and addition. According to some embodiments, the modification is a substitution. According to some embodiments, the peptide analog has at least 85%, at least 90% or at least 95% sequence identity to the original peptide. According to one embodiment, the analog has from about 70% to about 95%, about 80% to about 90% or about 85% to about 95% sequence identity to the original peptide. According to one embodiment, the analog has from about 85% to about 99% sequence identity to the original sequence. According to one embodiment, the analog has from about 90% to about 95% sequence identity to the original sequence. According to some embodiments, the analog of the present invention comprises the sequence of the original peptide in which 1, 2, 3, 4, or 5 substitutions were made. According to one embodiment, the substitution is a conservative substitution. According to any one of the embodiments of the present invention the analog is a functional analog, i.e., has the same function as the original sequence. According to some embodiments, the analog of the ABD or of scFv binds specifically STn and the original ABD or scFv. The term “analog” when referring to the sequence of the ABD of the present invention, to VH or VL domains contemplates that the modifications are not in the CDR region. Thus, in some embodiments, the present invention provides analogs of VH and/or VL domains, wherein the none of the amino acid alterations is not in the CDRs of the VH and/or VL. In other words, the analogs of VH and/or VL domains contemplate analog in which amino acid modifications are in frameworks regions only. According to some embodiments, the analog of ABD comprising the analog sequences of the VH and/or the VL of the present invention specifically binds to Sialyl Tn glycan.

According to some embodiments, the VH and the VL domains of the ABD of the CAR of the present invention are linked by a spacer to form a single chain variable fragment (scFv). The terms “linker” or “spacer” in the context of CAR refer to any peptide capable of connecting two domains of the ABD or CAR or two distinguishable sections of the CAR such as variable domains with its length depending on the kinds of variable domains to be connected. According to some embodiments, the spacer comprises an amino acid sequence comprising from 1 to 10 repetitions of amino acid sequence SEQ ID NO: 9. According to some embodiments, the spacer comprises 2, 3, 4, 5, or 6 repetitions of amino acid sequence SEQ ID NO: 9. According to one embodiment, the spacer comprises amino acid sequence comprising 3 repetitions of amino acid sequence SEQ ID NO: 9. According to one embodiment, the spacer comprises amino acid sequence SEQ ID NO: 10. According to some embodiments, the antigen binding domain of the CAR according to the present invention comprises amino acid sequence SEQ ID NO: 11 or an analog thereof having at least 90% sequence identity to said sequence. According to any one of the above embodiments, the analog comprises amino acid alternations in framework regions only and devoid of alternations in the CDRs.

The CAR of the present invention comprises a transmembrane domain (TM domain), one or more costimulatory domains and an activation domain.

In one embodiment of the invention, the CAR includes a transmembrane domain that comprises a transmembrane domain of a protein selected from the group consisting of the alpha, beta or zeta chain of the T-cell receptor, CD28, CD3 epsilon, CD45, CD4, CD5, CD8, CD9, CD16, CD22, CD33, CD37, CD64, CD80, CD86, CD134, CD137 and CD154 on an analog thereof. According to one embodiment, the TM domain is a TM domain of a receptor selected from CD28 and CD8, or an analog thereof having at least 85% amino acid identity to the original sequence.

In some embodiments of the invention, the CAR comprises a costimulatory domain, e.g., a costimulatory domain comprising a functional signaling domain of a protein selected from the group consisting of OX40, CD2, CD27, CD28, CD5, ICAM-1, LFA-1 (CD11a/CD18), ICOS (CD278), 4-1BB (CD137), an analog thereof and a combination thereof. According to one embodiment, the costimulatory domain is selected from a costimulatory domain of a protein selected from CD28, 4-1BB, OX40, an analog thereof having at least 85% amino acid identity to the original sequence, and any combination thereof. According to some embodiments, the CAR of the present invention comprises two or more costimulatory domains. According to one embodiment, the CAR comprises costimulatory domains of CD28 and 4-1BB.

According to one embodiment, the TM domain and the costimulatory domain of the CAR are both derived from CD28. According to one embodiment, the TM domain and the costimulatory domain have amino acid sequence SEQ ID NO: 12. According to another embodiment, the TM domain and the costimulatory domain have an amino acid sequence which is an analog of SEQ ID NO: 12 having at least 85% amino acid identity to SEQ ID NO: 17.

According to some embodiments, the antigen binding domain is linked to the TM domain via a spacer. According to one embodiment, the spacer comprises amino acid sequence comprising from 1 to 6 repetitions, such as 1, 2, 3, 4, 5 or 6 repetitions, of amino acid sequence SEQ ID NO: 9. According to one embodiment, the spacer comprises amino acid sequence comprising 2 repetitions of amino acid sequence SEQ ID NO: 9. According to another embodiment, the spacer comprises amino acid SEQ ID NO: 10.

According to any one of the above embodiments, the CAR comprises an activation domain selected from FcRγ (gamma) and CD3-3 (CD3-zetta) activation domains, or any other sequence that contains an intracellular tyrosine activating motif (ITAM). According to one embodiment, the activation domain is FcRγ domain. According to one embodiment, FcRγ domain has amino acid sequence SEQ ID NO: 13 or an analog thereof having at least 85% amino acid identity to the original sequence. The terms “activation domain” and “signaling domain” may be used interchangeably.

The term “CD28” refers to cluster of differentiation 28 protein. In some embodiments, the CD28 is a human CD28.

The term “CD8” refers to cluster of differentiation 8 protein being a transmembrane glycoprotein and serving as a co-receptor for the T cell receptor. According to one embodiment, the CD8 is a human CD8.

The terms “ICOS” and “Inducible T-cell COStimulator” refer to CD278 which is a CD28-superfamily costimulatory molecule. According to one embodiment, the ICOS is a human ICOS.

The term “4-1BB” refers to a CD137 protein which is a member of the tumor necrosis factor receptor family and has costimulatory activity for activated T cells. According to one embodiment, 4-1BB is a human 4-1BB.

The terms “CD35”′ and “CD3-zetta” refer to a ξ(zetta) chain of CD3 (cluster of differentiation 3) T cell co-receptor participating in activation of both the cytotoxic and helper T cells. According to one embodiment, CD35 comprises an immunoreceptor tyrosine-based activation motif (ITAM). According to one embodiment, the CD3ξ is human CD3° C. CD32 is sometimes also referred as CD247.

The term “FcRγ” refers to Fc gamma receptors, which generate signals within their cells through ITAM. These are immunoglobulin superfamily receptors that are found on various innate as well as adaptive immune cells, where the extracellular part binds IgGs the activation signal is transduced through two ITAMs located on its cytoplasmic tail.

According to any one of the above embodiments, the CAR further comprises a leading peptide. According to one embodiment, the leading peptide is located N-terminally to the ABD. According to one embodiment, the leading peptide has amino acid sequence SEQ ID NO: 14 or an analog thereof having at least 85% amino acid identity.

The term “leader peptide”, “leading peptide”, “lead peptide”, “signaling peptide” and “signal peptide” are used herein interchangeably and refer to a peptide that translocates or prompts translocation of the target protein to cellular membrane.

According to some embodiments, the CAR of the present invention may further comprise a tag sequence. The term “tag” or “label” refers to a moiety which is attached, conjugated, linked or bound to, or associated with, a compound (for example a protein, peptide, amino acid, nucleic acid and/or carbohydrate) and which may be used as a means of, for example, identifying, detecting and/or purifying a compound. According to some embodiments, the tag is selected haemagglutinin tag, myc tag, poly-histidine tag, protein A, glutathione S transferase, Glu-Glu affinity tag, substance P. FLAG peptide, streptavidin (strep) binding peptide and human FC tag. According to some embodiments, the tags is a strep-tag.

According to some embodiments, the present invention provides a CAR comprising a scFv comprising an antigen binding domain that binds specifically STn, a TM selected from the TM of CD8 and CD28, a costimulatory domain selected from a costimulatory domain of a protein selected from the group consisting of OX40, CD28, 4-1BB (CD137), and combinations thereof, and an activation domain selected from FcRγ and CD3-3 activation domains. According to some embodiments, the CAR comprises an scFv comprising an amino acid sequence SEQ ID NO: 11, the TM of CD28, a costimulatory domain of CD28, 4-1BB or both, and an activation domain of FcRγ. According to some embodiments, the scFv comprises an analog of an amino acid sequence SEQ ID NO: 11 having at least 90% to said sequences.

According to one embodiment, the present invention provides a CAR comprising amino acid sequence SEQ ID NO: 15. According to another embodiment, the present invention provides a CAR comprising amino acid sequence SEQ ID NO: 16. According to one embodiment, the present invention provides a CAR consisting of amino acid sequence selected from SEQ ID NO: 15 and 16. According to some embodiments, the present invention provides a CAR comprising of consisting of amino acid sequence analog of the amino acid sequence selected from SEQ ID NO: 15 and 16 having at least 90% sequence identity said sequence.

According to another aspect, the present invention provides a nucleic acid molecule encoding the CAR according to any one of the above embodiments and aspects. All aspects, terms, definition and embodiments defined above are encompassed and apply herein as well. Thus, according to one embodiment, the present invention provides a nucleic acid molecule encoding a chimeric antigen receptor (CAR) comprising an antigen binding domain that binds specifically to Sialyl Tn glycan (STn), wherein the antigen binding domain comprises three complementarity determining regions (CDRs) of a heavy-chain variable domain (VH) having amino acid sequence as set forth in SEQ ID NO: 1 and three CDRs of a light-chain variable domain (VL) having amino acid sequence as set forth in SEQ ID NO: 2. According to some embodiments, ABD comprises a VH domain comprising an amino acid sequence ding amino acid sequence as set forth in SEQ ID NO: 1 or an analog thereof and the VL domain comprising amino acid sequence SEQ ID NO: 2 or an analog thereof, wherein the analog has at least 90% sequence identity to said sequence.

According to some embodiments, the nucleic acid molecule encodes an amino acid sequence selected from SEQ ID NO: 1, SEQ ID NO: 2 and both SEQ ID NOs: 1 and 2, or an analog thereof having at least 90% sequence identity to said sequence.

The term “nucleic acid molecule” refers to a single stranded or double stranded sequence (polymer) of deoxyribonucleotides or ribonucleotides. The terms “nucleic acid” and “polynucleotide” are used herein interchangeably. In addition, the polynucleotide includes analogues of natural polynucleotides, unless specifically mentioned. According to one embodiment, the nucleic acid may be selected from the group consisting of deoxyribonucleic acid (DNA), ribonucleic acid (RNA), peptide nucleic acid (PNA), locked nucleic acid (LNA), and analogues thereof, but is not limited thereto. The term encompasses DNA, RNA, single stranded or double stranded and chemical modifications thereof. According to one embodiment, the nucleic acid molecule is DNA.

According to some embodiments, the nucleic acid molecule is an isolated nucleic acid molecule. The term “isolated nucleic acid” as used herein denotes that the nucleic acid is essentially free of other cellular components with which it is associated in the cell. It can be, for example, a homogeneous state and may be dry or in the state of a solution, such as aqueous solution. Purity and homogeneity are typically determined using analytical chemistry techniques such as polyacrylamide gel electrophoresis or high-performance liquid chromatography. The term “encoding” refers to the ability of a nucleotide sequence to code for one or more amino acids. The term does not require a start or stop codon. An amino acid sequence can be encoded in any one of six different reading frames provided by a polynucleotide sequence and its complement.

The terms “homolog” “variant”, “DNA variant”, “sequence variant” and “polynucleotide variant” are used herein interchangeably and refer to a polynucleotide such as DNA having at least 70% sequence identity to the polynucleotide from which it is derived (parent polynucleotide). The variant may include mutations such as deletion, addition or substitution such that the mutations do not change the open reading frame and the polynucleotide encodes a peptide or a protein having substantially similar structure and function as a peptide or a protein encoded by the parent polynucleotide. Thus, according the variant encodes to a polypeptide or protein that have the same function as the protein polypeptide or protein encoded by the original polynucleotide. In any occasion, no alternations are introduced in CDRs of the encoded sequences due to mutations in the variant. According to some embodiments, the variants are conservative variants. The term “conservative variants” as used herein refers to variants in which a change of one or more nucleotides in a given codon position results in no alteration in the amino acid encoded at that position. Thus, the peptide or the protein encoded by the conservative variants has 100% sequence identity to the peptide or the protein encoded by the parent polynucleotide. According to some embodiments, the variant is a non-conservative variant encoding to a peptide or a protein being a conservative analog of the peptide of the protein encoded by the parent polynucleotide. According to some embodiments, the variant has at least 75%, at least 80% at least 85%, at least 90%, at least 95%, at least 98% or at least 99% sequence identity to the original nucleic acid sequence. According to one embodiment, the variant is a conservative variant.

According to some embodiments, the nucleic acid molecule of the present invention comprises a nucleic acid sequence selected from SEQ ID NO: 17, SEQ ID NO: 18, a variant of SEQ ID NO: 17 or 18 having at least 95% sequence identity to the sequence(s), and a combination thereof.

According to some embodiments, the nucleic acid molecule encodes the amino acid sequence SEQ ID NO: 11 or an analog thereof having at least 90% sequence identity. According to other embodiments, the nucleic acid molecule comprises the nucleic acid sequence SEQ ID NO: 20 or a variant thereof having at least 95% sequence identity to said sequence.

According to any of the above embodiments, the nucleic acid molecule further encodes a TM domain, a stimulation domain and an activating domain of the T cell receptor. According to any of the above embodiments, the nucleic acid molecule further encodes an amino acid sequence selected from SEQ ID NOs: 12, 13, 14, an analog thereof, and any combination thereof. According to some embodiments, the nucleic acid comprises a nucleic acid sequence selected from SEQ ID NO: 22, 23, 24, a variant thereof having at least 95% sequence identity to said sequence(s), and a combination thereof.

According to some embodiments, the nucleic acid molecule encodes amino acid sequence SEQ ID NO: 15. According to other embodiments, the nucleic acid molecule encodes amino acid sequence SEQ ID NO: 16. According to further embodiments, the nucleic acid molecule encodes an analog of an amino acid sequence the selected from SEQ ID NO: 15 and SEQ ID NO: 16, the analog having at least 90% sequence identity to said sequence.

According to some embodiments, the nucleic acid molecule of the present intention comprises a nucleic acid sequence selected from SEQ ID NO: 25, SEQ ID NO: 26, and a variant thereof having at least 90% sequence identity to the original sequence.

According to any one of the aspects and embodiments of the invention, when referring to CAR or ABD the terms “comprising the amino acid sequence set forth in SEQ ID NO: X”, “comprising SEQ ID NO: X” and “having SEQ ID NO: X” are used herein interchangeably. The terms “consisting of the amino acid sequence set forth in SEQ ID NO: X”, “consisting of SEQ ID NO: X” and “of SEQ ID NO: X” are used herein interchangeably. The same rule holds for nucleic acid sequence. Thus, the terms “nucleic acid comprising the nucleic acid sequence set forth in SEQ ID NO: X”, “nucleic acid comprising SEQ ID NO: X” and “nucleic acid having SEQ ID NO: X” are used herein interchangeably. The terms “nucleic acid consisting of the nucleic acid sequence set forth in SEQ ID NO: X”, “nucleic acid consisting of SEQ ID NO: X” and “nucleic acid of SEQ ID NO: X” are used herein interchangeably. The terms “comprising”, “comprise(s)”, “include(s)”, “having”, “has” and “contain(s),” are used herein interchangeably and have the meaning of “consisting at least in part of”. When interpreting each statement in this specification that includes the term “comprising”, features other than that or those prefaced by the term may also be present. Related terms such as “comprise” and “comprises” are to be interpreted in the same manner. The terms “have”, “has”, having” and “comprising” also encompasses the meaning of “consisting of” and “consisting essentially of”, and may be substituted by these terms. Thus, according to any aspect or embodiment of the present invention, the statement such as VH or VL comprising amino acid sequence X encompasses also the meaning that the VH or VL consisting of amino acid sequence X.

According to another aspect, the present invention provides a nucleic acid construct comprising the nucleic acid molecule of the present invention, operably linked to a promoter. Any one of the above terms, definitions, and embodiments are encompassed and apply herein as well. Thus, according to one embodiment, the nucleic acid construct comprises a nucleic acid molecule comprising nucleic acid sequence SEQ ID NOs: 25 or a variant thereof having at least 95% sequence identity to the original sequence(s) operably bound to a promoter. According to another embodiment, the nucleic acid construct comprises a nucleic acid molecule comprising nucleic acid sequence SEQ ID NOs: 26 or a variant thereof having at least 95% sequence identity to the original sequence(s) operably bound to a promoter.

The term “nucleic acid construct” as used herein refers to an artificially constructed segment of a nucleic acid molecule. It can be an isolate or integrated into another DNA molecule. Accordingly, a “recombinant nucleic acid construct” is produced by laboratory methods.

The terms “operably linked”, “operatively linked”, “operably encodes”, “operably bound” and “operably associated” are used herein interchangeably and refer to the functional linkage between a promoter and nucleic acid sequence, wherein the promoter initiates transcription of RNA corresponding to the DNA sequence. A heterologous DNA sequence is “operatively associated” with the promoter in a cell when RNA polymerase which binds the promoter sequence transcribes the coding sequence into mRNA which then in turn is translated into the protein encoded by the coding sequence.

The term “promoter” as used herein refers to a regulatory sequence that initiates transcription of a downstream nucleic acid. The term “promoter” refers to a DNA sequence within a larger DNA sequence defining a site to which RNA polymerase may bind and initiate transcription. A promoter may include optional distal enhancer or repressor elements. The promoter may be either homologous, i.e., occurring naturally to direct the expression of the desired nucleic acid, or heterologous, i.e., occurring naturally to direct the expression of a nucleic acid derived from a gene other than the desired nucleic acid. A promoter may be constitutive or inducible. A constitutive promoter is a promoter that is active under most environmental and developmental conditions. An inducible promoter is a promoter that is active under environmental or developmental regulation, e.g., upregulation in response to xylose availability. Promoters may be derived in their entirety from a native gene, may comprise a segment or fragment of a native gene, or may be composed of different elements derived from different promoters found in nature, or even comprise synthetic DNA segments. It is understood by those skilled in the art that different promoters may direct the expression of a gene in different tissues or cell types, or at different stages of development, or in response to different environmental or physiological conditions. It is further understood that the same promoter may be differentially expressed in different tissues and/or differentially expressed under different conditions.

According to another aspect, the present invention provides a vector comprising the nucleic acid molecule or nucleic acid construct of the present invention. Any one of the above terms, definitions, and embodiments are encompassed and apply herein as well. The terms “vector” and “expression vector” are used herein interchangeably and refer to any viral or non-viral vector such as plasmid, virus, retrovirus, bacteriophage, cosmid, artificial chromosome (bacterial or yeast), phage, binary vector in double or single stranded linear or circular form, or nucleic acid, the sequence which is able to transform host cells and optionally capable of replicating in a host cell. The vector may be integrated into the cellular genome or may exist extrachromosomally (e.g., autonomous replicating plasmid with an origin of replication). The vector may contain an optional marker suitable for use in the identification of transformed cells, e.g., tetracycline resistance or ampicillin resistance. A cloning vector may or may not possess the features necessary for it to operate as an expression vector. Any vector known in the art is envisioned for use in the practice of this invention. According to other embodiments, the vector is a virus, e.g. a modified or engineered virus. The modification of a vector may include mutations, such as deletion or insertion mutation, gene deletion or gene inclusion. In particular, a mutation may be done in one or more regions of the viral genome. Such mutations may be introduced in a region related to internal structural proteins, replication, or reverse transcription function. Other examples of vector modification are deletion of certain genes constituting the native infectious vector such as genes related to the virus' pathogenicity and/or to its ability to replicate. Any virus can be attenuated by the methods disclosed herein. According to some embodiments, the vector is a virus selected from lentivirus, adenovirus, modified adenovirus and retrovirus. In one particular embodiment, the vector is lentivirus. According to some embodiments, the vector encodes an amino acid sequence selected from SEQ ID NOs: 1, 2, 11, 15, 16 and an analog thereof having at least 90% sequence identity to said sequence. According to other embodiments, the vector comprises a nucleic acid sequence selected from SEQ ID NOs: 17, 18, 20, 25, 26 and a variant thereof having at least 90% sequence identity to said sequence.

According to another aspect, the present invention provides a cell comprising the CAR, the nucleic acid molecule, the nucleic acid construct and/or the vector of the present invention. Any one of the above aspects, terms, definitions, and embodiments are encompassed and apply herein as well. According to some embodiment, the cell is selected from a bacterial, fungi (such as yeast) and mammalian cell.

According to some embodiments, the present invention provides a cell comprising the CAR of the present invention having amino acid sequence SEQ ID NO: 15. According to some embodiments, the present invention provides a cell comprising the CAR of the present invention having amino acid sequence SEQ ID NO: 16. According to some embodiments, the present invention provides a cell comprising the CAR of the present invention consisting of amino acid sequence selected from SEQ ID NO: 15 and 16. According to some embodiment, the present invention provides a cell comprising a nucleic acid molecule comprising a nucleic acid sequence selected from SEQ ID NO: 25 and 26.

According to some embodiments, the cell is a mammalian cell. According to another embodiment, the cell is a human cell. According to some embodiment, the cell is lymphocyte. According to some embodiments, the cell is a T cell. According to some embodiments, the cell is a natural killer (NK) cell.

The term “T cell” refers to a type of white blood cell that can be distinguished from other white blood cells by the presence of a T cell receptor on the cell surface. There are several subsets of T cells, including, but not limited to, T helper cells (a.k.a. Tx cells or CD4+ T cells) and subtypes, including T_{H}1, T_{H}2, T_{H}3, T_{H}17, T_{H}9, and T_{FH} cells, cytotoxic T cells (i.e., Tc cells, CD8+ T cells, cytotoxic T lymphocytes, T-killer cells, killer T cells), memory T cells and subtypes, including central memory T cells (T_{CM} cells), effector memory T cells (T_{EM} and T_{EMRA} cells), and resident memory T cells (T_{RM} cells), regulatory T cells (a.k.a. T_{reg} cells or suppressor T cells) and subtypes, including CD4+FOXP3⁺T_{reg} cells, CD4⁺FOXP3⁻ T_{reg} cells, Tr1 cells, Th3 cells, and T_{reg} 17 cells, natural killer T cells (a.k.a. NKT cells), mucosal associated invariant T cells (MAITs), and gamma delta T cells (γδ T cells), including Vγ9/Vδ2 T cells. Any one or more of the aforementioned or unmentioned T cells may be the target cell type for a method of use of the invention. According to some embodiments, the cells are T cells. According to some embodiments, the T-cells are selected from memory, regulatory, helper or natural killer T-cells. According to some embodiments, the T cell is selected are from CD4+ T-cell and a CD8+ T-cell. According to some embodiments, the T cell are CD4+ T-cell and a CD8+ T-cell. According to some embodiments, the cells are NK cells. According to some embodiments, the cells are NK T-cells.

According to some embodiments, the present invention provides T-cell comprising the CAR of the present invention comprising amino acid sequences SEQ ID NO: 1 and 2. According to some embodiments, the present invention provides T-cell comprising the CAR of the present invention having amino acid sequence selected from SEQ ID NO: 15 and 16. According to some embodiments, the cell expresses or capable of expressing the CAR of the present invention. Thus, according to some embodiments, the present invention provides a

T-cell genetically modified to express the CAR of the present invention. According to one embodiment, the present invention provides a T-cell genetically modified to express or expressing a CAR comprising amino acid sequence SEQ ID NO: 15. According to another embodiment, the present invention provides a T-cell genetically modified to express or expressing a CAR comprising amino acid sequence SEQ ID NO: 16. According to another embodiment, the present invention provides a T-cell comprising, expressing or capable of expressing a CAR comprising an analog having at least 90% sequence identity to amino acid sequence selected from SEQ ID NO: 15 and 16. The term “capable of expressing” refers to cells comprising a nucleic acid encoding the CAR of the present invention and are engineered to express it constantly or upon in induction.

According to some embodiments, the cell, such as T-cell comprises the nucleic acid molecule encoding the CAR of the present invention. According to other embodiments, the cell, such as T-cell comprises the nucleic acid construct comprising nucleic acid molecule encoding the CAR of the present invention. According to a further embodiment, the present invention provides a cell comprising a vector comprising the nucleic acid construct or molecule encoding the CAR of the present invention. According to such embodiments, the T-cell is capable of expressing or expresses the CAR of the present invention. According to some embodiment, the present invention provides a T-cell comprising a nucleic acid molecule comprising a nucleic acid sequence selected from SEQ ID NO: 25 and 26. According to some embodiments, the T-cell comprising the nucleic acid of the present invention express or capable of expressing the CAR of the present invention, e.g., a CAR comprising an amino acid sequence selected from SEQ ID NO: 15 and 16.

According to some embodiments, the present invention provides a plurality of the cells according to any one of the above embodiments. According to some embodiments, the present invention provides a plurality of T cell cells according to any one of the above embodiments.

According to another aspect, the present invention provides a composition comprising a plurality of CARs or cells of the present invention and a carrier. According to some embodiments, the present invention provides a composition comprising a plurality of CARs of the present invention. All aspects, terms, definitions and embodiments defined above are encompassed and apply herein as well. The term “carrier” includes as a class any compound or composition useful in facilitating storage, stability, and use, including, without limitation, suitable vehicles, solvents, protectants, diluents, excipients, pH modifiers, salts, rheology modifiers, surfactants, emulsifiers, surfactants, preservatives, chelating agents, and water. According to some embodiments, the composition is a pharmaceutical composition. According to some embodiments, the present invention provides a pharmaceutical composition comprising a plurality of CARs of the present invention and a pharmaceutically acceptable carrier. According to other embodiments, the present invention provides a pharmaceutical composition comprising a plurality of cells of the present invention and a pharmaceutically acceptable carrier. The plurality of cells may also be referred to as a cell composition. According to some embodiments, the pharmaceutical composition comprises a plurality of T-cells expressing the CAR of the present invention having amino acid sequence SEQ ID NO: 15. According to other embodiments, the pharmaceutical composition comprises a plurality of T-cells expressing the CAR of the present invention having amino acid sequence SEQ ID NO: 16. According to some embodiments, the pharmaceutical composition comprises a plurality of T-cells capable of expressing the CAR of the present invention having amino acid sequence selected from SEQ ID NO: 15 and 16. According to some embodiments, the pharmaceutical composition comprises a plurality of T-cells expressing or capable of expressing the CAR of the present invention being an analog of amino acid sequence selected from SEQ ID NO: 15 and 16, wherein the analog has at least 90% sequence identity to said sequences. According to another embodiment, the present invention provides the pharmaceutical compositions comprising a plurality of T-cells comprising the nucleic acid molecule, construct or vector and capable of expressing the CAR of the present invention. According to another embodiment, the nucleic acid molecule, construct or vector comprises a nucleic acid sequence selected from SEQ ID NO: 25 and 26 and are capable of expressing the CAR of the present invention. According to some embodiments, the T-cells are CD8+ T-cells. According to other embodiments, the T-cells are CD4+ T-cells. According to some embodiments, the T-cells are a combination of CD4+ and CD8+ cells.

The term “pharmaceutical composition” as used herein refers to a composition comprising at least one active agent as disclosed herein, e.g. CAR or CAR T-cells, formulated together with one or more pharmaceutically acceptable carriers.

Formulations of the pharmaceutical composition may be adjusted according to applications. In particular, the pharmaceutical composition may be formulated using a method known in the art so as to provide a rapid, continuous or delayed release of the active ingredient after administration to mammals. For example, the formulation may be any one selected from among plasters, granules, lotions, liniments, lemonades, aromatic waters, powders, syrups, ophthalmic ointments, liquids and solutions, aerosols, extracts, elixirs, ointments, fluidextracts, emulsions, suspensions, decoctions, infusions, ophthalmic solutions, tablets, suppositories, injections, spirits, capsules, creams, troches, tinctures, pastes, pills, and soft or hard gelatin capsules.

The pharmaceutical compositions of the present invention may be prepared by conventional techniques, e.g., as described in Remington: The Science and Practice of Pharmacy, 19th Ed., 1995. The compositions may be in solid, semisolid or liquid form and may further include pharmaceutically acceptable fillers, carriers or diluents, and other inert ingredients and excipients. The compositions can be administered by any suitable route, e.g., orally, intravenously, parenterally, rectally or transdermally, the oral route being preferred. The dosage will depend on the state of the patient and will be determined as deemed appropriate by the practitioner.

The term “pharmaceutically acceptable carrier” or “pharmaceutically acceptable excipient” as used herein refers to any and all solvents, dispersion media, preservatives, antioxidants, coatings, isotonic and absorption delaying agents, surfactants, fillers, disintegrants, binders, diluents, lubricants, glidants, pH adjusting agents, buffering agents, enhancers, wetting agents, solubilizing agents, surfactants, antioxidants the like, that are compatible with pharmaceutical administration. The use of such media and agents for pharmaceutically active substances is well known in the art. The compositions may contain other active compounds providing supplemental, additional, or enhanced therapeutic functions, solid carriers or excipients such as, for example, lactose, starch or talcum or liquid carriers such as, for example, water, fatty oils or liquid paraffins.

Solutions or suspensions used for parenteral, intradermal, or subcutaneous application typically include the following components: a sterile diluent such as water for injection, saline solution, fixed oils, polyethylene glycols, glycerin, propylene glycol (or other synthetic solvents), antibacterial agents (e.g., benzyl alcohol, methyl parabens), antioxidants (e.g., ascorbic acid, sodium bisulfite), chelating agents (e.g., ethylenediaminetetraacetic acid), buffers (e.g., acetates, citrates, phosphates), and agents that adjust tonicity (e.g., sodium chloride, dextrose). The pH can be adjusted with acids or bases, such as hydrochloric acid or sodium hydroxide, for example. The parenteral preparation can be enclosed in ampules, disposable syringes or multiple dose glass or plastic vials.

Pharmaceutical compositions adapted for parenteral administration include, but are not limited to, aqueous and non-aqueous sterile injectable solutions or suspensions, which can contain antioxidants, buffers, bacteriostats and solutes that render the compositions substantially isotonic with the blood of an intended recipient. Such compositions can also comprise water, alcohols, polyols, glycerin and vegetable oils, for example. Extemporaneous injection solutions and suspensions can be prepared from sterile powders, granules and tablets. Such compositions preferably comprise a therapeutically effective amount of a compound of the invention and/or other therapeutic agent(s), together with a suitable amount of carrier so as to provide the form for proper administration to the subject.

According to some embodiments, the composition is formulated for a parenteral administration. According to one embodiment, the composition is formulated for subcutaneous, intraperitoneal (IP), IM, IV and intratumor administration. According to other embodiments, the composition is formulated as a solution such as a sterile solution for injection.

According to any one of the above embodiments, the pharmaceutical composition of the present invention is for use in treating cancer. According to some embodiments, the use comprises administering the pharmaceutical composition to a subject. According to some embodiments, the cancer is cancer overexpressing STn glycan. According to some embodiments, the cancer cells present STn glycan. The term “cancer” comprises cancerous diseases or a tumor being treated or prevented that is selected from the group comprising. but not limited to, mammary carcinomas, melanoma, skin neoplasms, lymphoma, leukemia, gastrointestinal tumors, including colon carcinomas, stomach carcinomas, pancreas carcinomas, colon cancer, small intestine cancer, ovarian carcinomas, cervical carcinomas, lung cancer, prostate cancer, kidney cell carcinomas and/or liver cancer and/or metastases breast, colorectal, oropharyngeal cancer, head and neck and gallbladder cancer, and squamous cell carcinoma. According to another embodiment, the cancer is selected from lung adenocarcinoma, pancreatic adenocarcinoma, colon adenocarcinoma, Her-2 negative/positive breast carcinoma and pharynx squamous cell carcinoma. The cancer can also be cancers of blood and bone marrow as leukemia, lymphoma and myeloma. According to some embodiments, the cancer is selected from carcinoma and lymphoma. According to some embodiments, cancer is selected from endometrial carcinoma, ovarian carcinoma, prostate adenocarcinoma, seminoma, diffuse type gastric adenocarcinoma, pancreatic and colon adenocarcinomas, lung adenocarcinoma and mantle cell lymphoma. According to some embodiments, cancer is pancreatic colon. According to some embodiments, cancer is colon adenocarcinoma. According to some embodiments, cancer is ovarian carcinoma. According to some embodiments, cancer is prostate adenocarcinoma.

The term “treating” a condition or patient refers to taking steps to obtain beneficial or desired results, including clinical results. Beneficial or desired clinical results include, but are not limited to, or ameliorating abrogating, substantially inhibiting, slowing or reversing the progression of a disease, condition or disorder, substantially ameliorating or alleviating clinical or esthetical symptoms of a condition, substantially preventing the appearance of clinical or esthetical symptoms of a disease, condition, or disorder, and protecting from harmful or annoying symptoms. Treating further refers to accomplishing one or more of the following: (a) reducing the severity of the disorder; (b) limiting development of symptoms characteristic of the disorder(s) being treated; (c) limiting worsening of symptoms characteristic of the disorder(s) being treated; (d) limiting recurrence of the disorder(s) in patients that have previously had the disorder(s); and/or (e) limiting recurrence of symptoms in patients that were previously asymptomatic for the disorder(s).

The term “treating cancer” as used herein should be understood to e.g. encompass treatment resulting in a decrease in tumor size; a decrease in rate of tumor growth; stasis of tumor size; a decrease in the number of metastasis; a decrease in the number of additional metastasis; a decrease in invasiveness of the cancer; a decrease in the rate of progression of the tumor from one stage to the next; inhibition of tumor growth in a tissue of a mammal having a malignant cancer; control of establishment of metastases; inhibition of tumor metastases formation; regression of established tumors as well as decrease in the angiogenesis induced by the cancer, inhibition of growth and proliferation of cancer cells and so forth. The term “treating cancer” as used herein should also be understood to encompass prophylaxis such as prevention as cancer reoccurs after previous treatment (including surgical removal) and prevention of cancer in an individual prone (genetically, due to life style, chronic inflammation and so forth) to develop cancer. As used herein, “prevention of cancer” is thus to be understood to include prevention of metastases, for example after surgical procedures or after chemotherapy.

According to some embodiments, the use comprises administering the pharmaceutical composition to a subject. According to any one of the above embodiments, the composition of the present invention is administered as known in the art. According to one embodiment, the composition is parenterally administered, e.g. IP, IV, IM, SC or intratumorally. According to some embodiments, the pharmaceutical composition is administered via infusion.

The terms “administering” or “administration of” a substance, a compound or an agent to a subject are used herein interchangeably and refer to an administration mode can be carried out using one of a variety of methods known to those skilled in the art. For example, a compound or an agent can be administered, intravenously, arterially, intradermally, intramuscularly, intraperitoneally, intravenously, subcutaneously, ocularly, sublingually, orally (by ingestion), intranasally (by inhalation), intraspinally, intracerebrally, and transdermally (by absorption, e.g., through a skin duct). A compound or agent can also appropriately be introduced by rechargeable or biodegradable polymeric devices or other devices, e.g., patches and pumps, or formulations, which provide for the extended, slow or controlled release of the compound or agent. Administering can also be performed, for example, once, a plurality of times, and/or over one or more extended periods. According to some embodiments, the composition is administered 1, 2, 3, 4, 5 or 6 times a day. According to other embodiments, the composition is administered 1, 2, 3, 4, 5 or 6 times a month. In some embodiments, the administration includes both a direct administration, including self-administration, and indirect administration, including the act of prescribing a drug. For example, as used herein, a physician who instructs a patient to self-administer a drug, or to have the drug administered by another and/or who provides a patient with a prescription for a drug is administering the drug to the patient. According to one embodiment, the pharmaceutical composition is parenterally administered. The term “parenteral” refers to subcutaneous, intracutaneous, intravenous, intramuscular, intraarticular, intraarterial, intrasynovial, intrasternal, intrathecal, intralesional, intraperitoneal and intracranial injection, as well as various infusion techniques.

According to some embodiments, the pharmaceutical composition of the present invention comprising the CAR T-cells of the present invention is co-administered with an additional anti-tumor therapy including but not limited to anticancer drugs, radiotherapy, immunotherapy and surgery. According to some embodiments, the pharmaceutical composition is co-administered with another anti-cancer drug. According to some embodiments, the therapeutic agents suitable in an anti-neoplastic composition for treating cancer include, but not limited to, chemotherapeutic agents, radioactive isotopes, toxins, cytokines such as interferons, immunostimulating agents, immunomodulating agents and antagonistic agents targeting cytokines, cytokine receptors or antigens associated with tumor cells. In some embodiments, the anti-cancer agent is a chemotherapeutic.

The term “co-administration” encompasses the administration of a first and second agent in an essentially simultaneous manner, such as in a single dosage form, e.g., a capsule or tablet having a fixed ratio of first and second amounts, or in multiple dosage forms for each. The agents can be administered in a sequential manner in either order. When co-administration involves the separate administration of each agent, the agents are administered sufficiently close in time to have the desired effect (e.g., complex formation). The term “anti-cancer”, “anti-neoplastic” and “anti-tumor” when referred to a compound, an agent or a moiety are used herein interchangeably and refer to a compound, drug, antagonist, inhibitor, or modulator such as immunomodulatory having anticancer properties or the ability to inhibit or prevent the growth, function or proliferation of and/or causing destruction of cells,” and in particular tumor cells. Therapeutic agents suitable in an anti-neoplastic composition for treating cancer include, but not limited to, chemotherapeutic agents, radioactive isotopes, toxins, cytokines such as interferons, immunostimulating agents, immunomodulating agents and antagonistic agents targeting cytokines, cytokine receptors or antigens associated with tumor cells. In some embodiments, an anti-cancer agent is a chemotherapeutic.

According to yet another aspect, the present invention provides use of CARs of the present invention of cells comprising the CAR of the present invention in the preparation of a medicament for treating cancer. According to other embodiments, the present invention provides use of the CARs of the present invention in the preparation of a medicament for treating cancer. According to other embodiments, the present invention provides use of a plurality of T cells comprising, expressing or designed to express the CARs of the present invention in the preparation of a medicament for treating cancer.

According to another aspect, the present invention provides a method for treating cancer in a subject in need thereof comprising administering to the subject a therapeutically effective amount of cells or the pharmaceutical composition of the present invention. According to some embodiments, the cells are engineered cells expressing the CAR of the present invention. All aspects and embodiments defined above apply herein as well. According to other embodiments, the cells are engineered to express the CAR of the present invention. According to some embodiments, the cells are CAR T-cells. According to some embodiments, the cells are NK cells comprising CAR the CAR of the present invention. The term “therapeutically effective amount” of a drug or agent is an amount of a drug or an agent that, when administered to a subject will have the intended therapeutic effect. The full therapeutic effect does not necessarily occur by administration of one dose, and may occur only after administration of a series of doses. Thus, a therapeutically effective amount may be administered in one or more administrations. The precise effective amount needed for a subject will depend upon, for example, the subject's size, health and age, the nature and extent of the cognitive impairment, and the therapeutics or combination of therapeutics selected for administration, and the mode of administration. The skilled person can readily determine the effective amount for a given situation by routine experimentation.

According to another aspect, the present invention provides a use of CAR T-cells of the present invention for preparation of a medicament for treating cancer.

According to another aspect, the present invention provides a method of preparation of the T-cells of the present invention. According to one embodiment, the present invention provides a method of preparation of T-cells genetically modified to express the CARs of the present invention. According to some embodiments, the method comprises transfecting of T-cells with the DNA construct of the present invention.

All previous definitions, terms, aspects and embodiments apply and are encompassed herein as well.

The terms “transfection”, “transduction”, “transfecting” or “transducing” can be used interchangeably and are defined as a process of introducing a nucleic acid molecule to a cell. Nucleic acids are introduced to a cell using non-viral or viral-based methods. The nucleic acid molecules may be gene sequences encoding complete proteins or functional portions thereof. Non-viral methods of transfection include any appropriate transfection method that does not use viral DNA or viral particles as a delivery system to introduce the nucleic acid molecule into the cell. Exemplary non-viral transfection methods include calcium phosphate transfection, liposomal transfection, nucleofection, sonoporation, transfection through heat shock, magnetifection and electroporation. In some embodiments, the nucleic acid molecules are introduced into a cell using electroporation following standard procedures well known in the art. For viral-based methods of transfection any useful viral vector may be used in the methods described herein. Examples for viral vectors include, but are not limited to retroviral, adenoviral, lentiviral and adeno-associated viral vectors. In some embodiments, the nucleic acid molecules are introduced into a cell using a retroviral vector following standard procedures well known in the art.

According to one embodiment, the T-cells are CD4+ T-cells. According to another embodiment, the T-cells are CD8+ cells. According to one embodiment, the T-cells are a combination of CD4+ and CD8+ cells.

According to one embodiment, the method comprises transducing T cells with at least one DNA construct encoding the CAR comprising an amino acid sequence selected from SEQ ID NO: 15, 16 or an analog thereof as defined as defined in the present invention. According to some embodiments, the DNA construct comprises a nucleic acid sequence selected from SEQ ID NO: 25, 26 and a variant thereof as defined in the present invention.

According to any one of the above embodiments, the transduction is performed using a viral vector selected from retroviral, adenoviral, lentiviral and adeno-associated viral vectors.

According to some embodiments, the vector may contain an optional marker suitable for use in the identification of the transformed cells.

The term “consisting essentially of” means that the composition or component may include additional ingredients, but only if the additional ingredients do not materially alter the basic and novel characteristics of the claimed compositions or methods.

The terms “comprising”, “comprise(s)”, “include(s)”, “having”, “has” and “contain(s),” are used herein interchangeably and have the meaning of “consisting at least in part of”. When interpreting each statement in this specification that includes the term “comprising”, features other than that or those prefaced by the term may also be present. Related terms such as “comprise” and “comprises” are to be interpreted in the same manner. The terms “have”, “has”, having” and “comprising” may also encompass the meaning of “consisting of” and “consisting essentially of”, and may be substituted by these terms. The term “consisting of” excludes any component, step or procedure not specifically delineated or listed. The term “consisting essentially of” means that the composition or component may include additional ingredients, but only if the additional ingredients do not materially alter the basic and novel characteristics of the claimed compositions or methods.

Having now generally described the invention, the same will be more readily understood through reference to the following examples, which are provided by way of illustration and are not intended to be limiting of the present invention.

### EXAMPLES

### Materials and Methods

### Cloning of RA0

The variable domains of RA0 antibody were cloned into p3BNC plasmids containing the constant regions of human IgG1 Fc, then expressed as a full-length chimeric antibody in HEK-293A cells (ATCC; CRL-1573).

### Sialoglycan Microarray Fabrication.

Arrays were fabricated with NanoPrint LM-60 Microarray Printer (Arrayit) on epoxide-derivatized slides (Corning 40044) with 16 sub-array blocks on each slide. Glycoconjugates were distributed into one 384-well source plates using 4 replicate wells per sample and 8 μl per well (Version 3.1). Each glycoconjugate was prepared at 100 μM in an optimized print buffer (300 mM phosphate buffer, pH 8.4). To monitor printing quality, replicate-wells of human IgG (80, 40, 20, 10, 5, 0.25 ng/μl in PBS+10% glycerol) and AlexaFlour-555-Hydraside (Invitrogen A20501MP, at 1 ng/μl in 178 mM phosphate buffer, pH 5.5) were used for each printing run. The arrays were printed with four 946MP3 pins (5 μm tip, 0.25 μl sample channel, ˜100 μm spot diameter; Arrayit). Each block (sub-array) has 20 spots/row, 20 columns with spot to spot spacing of 275 μm. The humidity level in the arraying chamber was maintained at about 70% during printing. Printed slides were left on arrayer deck over-night, allowing humidity to drop to ambient levels (40-45%). Next, slides were packed, vacuum-sealed and stored at room temperature (RT) until used.

Sialoglycan microarray binding assay.

Slides were developed and analyzed. Slides were rehydrated with dH₂O and incubated for 30 min in a staining dish with 50° C. pre-warmed ethanolamine (0.05 M) in Tris-HCl (0.1 M, pH 9.0) to block the remaining reactive epoxy groups on the slide surface, then washed with 50° C. pre-warmed dH₂O. Slides were centrifuged at 200×g for three min then fitted with ProPlate™ Multi-Array 16-well slide module (Invitrogen) to divide into the sub-arrays (blocks). Slides were washed with PBST (0.1% Tween 20), aspirated and blocked with 200 μl/sub-array of blocking buffer (PBS/OVA, 1% w/v ovalbumin, in PBS, pH 7.3) for 1 hour at RT with gentle shaking. Next, the blocking solution was aspirated and 100 μl/block of purified mRAO antibody in 0.16 ng/μl diluted in PBS/OVA were incubated with gentle shaking for 2 hours at RT. Slides were washed three times with PBST, then with PBS for 2 min. Bound antibodies were detected by incubating with secondary detection diluted in PBS, 200 μl/block at RT for 1 hour, Cy3-goat anti mouse IgG 1.5 μg/ml (Jackson Immunoresearch). Slides were washed three times with PBST then with PBS for 10 min followed by removal from ProPlate™ Multi-Array slide module and immediately dipping in a staining dish with dH₂O for 10 min with shaking, then centrifuged at 200×g for 3 min. Dry slides immediately scanned.

### Array Slide Processing.

Processed slides were scanned and analyzed as described at 10 μm resolution with a Genepix 4000B microarray scanner (Molecular Devices) using 300 gain. Image analysis was carried out with Genepix Pro 6.0 analysis software (Molecular Devices). Spots were defined as circular features with a variable radius as determined by the Genepix scanning software. Local background subtraction was performed.

### Enzyme-Linked Immunosorbent Assay (ELISA)

Binding of RA0 anti-STn hIgG to STn was tested by ELISA. Costar 96-well were coated overnight at 4° C. with 0.25 μg STn-PAA-Biotin (Glycotech) in coating buffer (50 mM sodium carbonate-bicarbonate buffer, pH 9.5). RA0 antibodies at 0.2 ng/μl were pre-incubated with frec glycans, STn-PAA-Biotin, Neu5Acα2-3GalNAcα-PAA-Biotin, Tn-PAA-Biotin, SLe^{a}-PAA-Biotin (glycotech), for 2 h on ice. Wells were blocked for 1 h at RT with blocking buffer (PBS pH 7.3, 1% chicken ovalbumin (Sigma); PBS/OVA). After removal of blocking buffer, RA0-glycan mixture were added to triplicate wells at 100 μl/well then incubated at RT for 2 h. Wells were washed three times with PBST (PBS pH 7.3, 0.1% Tween-20), detection antibody was then added (100 μl/well, 1:7000 HRP-goat-anti-human IgG (H+L) diluted in PBS and incubated for 1 h at RT. After washing three times with PBST, wells were developed with 0.5 mg/ml O-phenylenediamine in citrate-PO₄ buffer, pH 5.5, reaction was stopped with H₂SO₄ and absorbance was measured at a 490 nm wavelength on a SpectraMax M3 (Molecular Devices).

### Tissue Microarray Immunohistochemical Staining

The cloned RA0 human IgG1 antibody was biotinylated using the EZ-Link biotinylation Kit (Micro Sulfo-NHS-SS-Biotin; Pierce, Rockford, IL) according to the manufacturer's instructions, then human cancers tissue microarray (TMA) slides (BioSB CA, USA) consisting of twenty-three 2 mm cores formalin-fixed paraffin-embedded tissues were stained with this Bio-RA0-hIgG antibody. For this purpose, the slides were first deparaffinated by incubation in xylene (Merck) for 15 min twice, then rehydrated by sequential 2 min washes with a decreased percentage of ethanol in double-distilled H₂O solution (100%, 95%, 90%, 80%, 70%, 50%, DDW), then washed twice in DDW. For antigen unmasking, slides were incubated for 15 min with 95° ° C. pre-heated HIER T-EDTA buffer pH 9 (Zymo), then transferred to DDW for additional 15 min, followed by rinsing in PBS pH 7.4 once. Slides were then blocked for one hour at room temperature (RT) by incubating with blocking solution (PBS pH 7.4, 0.1% Tween, 1% chicken ovalbumin [Sigma]). Biotin/avidin blocking was performed using a kit (Zotal), according to manufacturer's instructions. Slides were rinsed briefly with PBS, then fixed with 4% paraformaldehyde (PFA) for 10 min in RT, washed with PBST (PBS pH 7.4, 0.1% Tween) for 1 min, and incubated with 10 ng/μl Bio-RA0-hIgG overnight at 4° C. in a humidified chamber. The next day, slides were washed in PBST for 5 min, twice, then incubated with freshly prepared 0.3% H₂O₂ in PBS for 15 min. After one wash with PBS pH7.4, slides were incubated with 1 μg/ml HRP-streptavidin in PBS (Jackson) for 30 minutes at RT, followed by three washes with PBS 5 min each, then developed with substrate (3,3′-diaminobenzidine tetrahydrochloride; DAB) for 3 min, followed by washing once with DDW for 1 min and mounting with PermaMounter (Bio-SB). Slides were screened with Nikon eclipse Ti microscope at ×10 magnification.

### Cell Lines and Culture Cell Lines

293T human embryonic kidney cells (ATCC; CRL-3216), FaDu pharynx squamous cell carcinoma cells (ATCC; HTB43), Raji, MEG-01, Capan-2, and packaging cell lines and PG13 (ATCC; CRL-10686) were cultured in DMEM supplemented with 10% fetal calf serum (FCS), 2 mM glutamine and 1 mM sodium pyruvate. Mouse lymphocytes were cultured in RPMI-1640 (Biological Industries) supplemented with 10% FCS, 2 mM glutamine. All media were supplemented with a mixed antibiotic solution containing penicillin (100 U/ml), streptomycin (100 μg/ml) and neomycin (10 μg/ml) (Bio-Lab). B16F10 were a kind gift from Professor Dan Peer at Tel Aviv University, cultured in DMEM supplemented with 10% fetal calf serum (FCS), 2 mM glutamine and a mixed antibiotic solution containing penicillin (100 U/ml), streptomycin (100 μg/ml). The cells were incubated in a humidified 37° C. incubator with 5% CO₂, except for the PG13, which were kept with 7.5% CO₂. All cells were verified to lack mycoplasma by PCR (HyLabs). The cells were frozen at low passage, and the number of passages after thawing was recorded. Cells were maintained in culture for no longer than 4 weeks, which corresponds to approximately 12 passages.

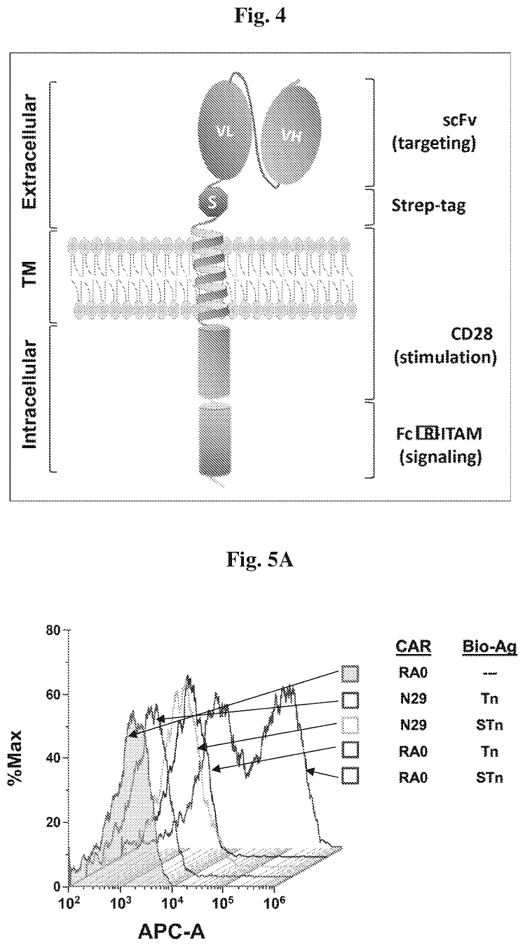

Construction of chimeric antigen receptor (CAR) and retroviral vector production The CAR construct contained a leader signal peptide, RA0 scFv (VH connected to the VL through 3×G₄S spacer), strep-tag, 2×G₄S spacer, human CD28 (hCD28 cytoplasmic, transmembrane and co-stimulation domains) followed by the human FcγR ITAM signaling domain (FIG. 4). Additional CAR constructs are prepared without strep-tag. Such CAR constructs comprise (i) a leader signal peptide, RA0 scFv 2×G₄S spacer, human CD28 (hCD28 cytoplasmic, transmembrane and co-stimulation domains) followed by the human FcγR ITAM signaling domain or (ii) a leader signal peptide, RA0 scFv (VH connected to the VL through 3×G₄S spacer), human CD28 (hCD28 cytoplasmic, transmembrane and co-stimulation domains) followed by the human CD3zeta or FcγR ITAM signaling domain.

VH has the amino acid sequence (SEQ ID NO: 1):
QVQLQESGPGLVAPSQSLSITCTVSGFSLISYGVSWVRQPPGKGLEWLG
VIWGDGSTNYHSTLISRLSINKDNSKSQVFLKLNSLQTDDTATYYCVGP
RFAYWGQGTLVTVSA
VL has the amino acid sequence (SEQ ID NO: 2):
QIVLTQSPAIMSASPGEKVTMTCSASSSVSYMHWYQQKSGTSPKRWIYD
TSKLTSGVPARFSGSGSGTSYSLTISSMEAEDAATYYCQQWSSNTLTFG
AGTKLELK

The sequence was cloned into the retroviral vector pMSGV1. In order to produce a stable packaging cell line, 293T cells were co-transfected with retroviral vector plasmids (Gag-Pol-VSV-G) and the plasmid of interest (pMSGV1-CAR T) using CaPO₄, as described (Globerson Levin et al., Cancer Immunol Res 2020 Oct. 2, 2020). Supernatants containing the retrovirus were collected 16 hours later and used to stably transduce the amphotropic PG13 packaging cells. Cells were sorted by FACSort flow Cytometer (BD PharMingen) to achieve 100% RA0-CAR⁺-PG13 expressing cells, re-grown and frozen at −80° C.

### T Cell Transduction

T cell transduction was done as previously described (Globerson Levin et al., Cancer Immunol Res 2020 Oct. 2, 2020). Briefly, lymphocytes were isolated from the spleen of wild-type C57BL/6 mice. Splenocytes were activated in non-tissue culture-treated 6-well plates pre-coated with both anti-mouse CD3 (prepared in-house from hybridoma 2C11) and hamster anti-mouse CD28-UNLB (Southern Biotech) in RPMI+FCS+L−glu+P/S for 48 hours at 37° C. Activated lymphocytes were harvested, divided into two groups then co-cultured for 48 hours with 100 IU/ml IL-2 (untransduced cells) or for only one or two consecutive retroviral transductions in RetroNectin (Takara Shuzu Ltd.) that was pre-coated to non-tissue culture-treated 6-well plates supplemented with 100 IU/ml human IL-2 (Novartis Pharma GMbH). At the end of transduction, both untransduced and transduced T cells were cultured in RPMI+FCS+L-glu+P/S in the presence of 350 IU/ml IL-2 for 24-72 hours for in-vitro or in-vivo assays, respectively. Transduction efficiency was monitored by flow cytometry analysis using FITC-mouse-anti-strep-tag IgG1 (LSBio) according to manufacturer instructions.

### Stimulation Assays

For glycan stimulation assay, 24 wells plate was coated with 6.25 μg/well HRP-conjugated streptavidin (SA-HRP; Jackson) diluted in 0.5 ml of 50 mM sodium carbonate-bicarbonate buffer, pH 9.5 (coating buffer) and incubated overnight at 4° C. The following day, unbound SA-HRP was washed twice with 1 ml PBS. Then, 1.5625 μg/well STn-Biotin or STn-PAA-Bio (Glycotech) in 0.5 ml PBS and plate was incubated for one more night at 4ºC. The following day, unbound glycans were washed twice with 1 ml PBS. Then, 1 million cells/well of RA0 or UT mouse CAR-T in 1 ml RPMI+FCS+L-glu+P/S+beta mercaptoethanol+HEPES and plate was incubated for 16 hours at 37° C. The cell-free growth medium was collected and analyzed for IFN-γ, IL-2 and TNF-α production by ELISA using a mouse IFN-γ and TNF-α ELISA kit, according to the manufacturer's instructions (PeproTech).

For co-culturing stimulation assay, a total of 1×10⁶ untransduced or RA0 CAR transduced T cells were co-cultured with 0.5×10⁶ of cells (FaDu, Raji, Capan-2 or MEG-01) in 24-wells for 16 hours in a RPMI medium supplemented with 10% FCS, 2 mM glutamine and antibiotics. The cell-free growth medium was collected and analyzed for IFN-γ production by ELISA using a human IFN-γ ELISA kit, according to the manufacturer's instructions (R&D systems).

### Flow Cytometry

Cells were collected from plates using 10 mM EDTA. Cells were incubated with RA0 antibodies diluted in PBS+0.5% fish gelatin for 1 hour on ice, followed by incubation with Cy3 AffiniPure Goat Anti-Human IgG (H+L) (Jackson) diluted 1:100 in PBS+0.5% fish gelatin for 1 hour on ice. Fluorescence of cells were measured by CytoFLEX flow cytometry (Beckman Coulter).

### CDC Assay

For complement-dependent cytotoxicity (CDC) we used rabbit complement (Sigma). Cytotoxicity was evaluated by measuring lactate dehydrogenase (LDH) release using LDH Cytotoxicity Detection kit (Roche Applied Science) according to the manufacturer's instructions. All assays included maximum release control contains rabbit complement diluted 1:6 with 1% TritonX-100. For spontaneous release control, cells were incubated only with rabbit complement. Percentage cytotoxicity was calculated as: (test release-spontaneous release)/(maximum release-spontaneous release)×100. 2×10⁴ target Cells were incubated in triplicates with RA0 antibodies at 4 and 2 ng/μl for 1 hour on ice in 96-well round-bottom plates. Rabbit complement and triton were added and plates were incubated for 2 hours at 37° C. Then supernatants were collected and LDH release was determined.

### CAR T Glycan Specificity

5x 105 RA0-CAR T Cells and N29-CAR T Cells (served as irrelevant control CAR T cells; targeting ErbB2) were incubated with 1 μM biotinylated-polyacrylamide conjugated glycans (Glycotech; 6-8 glycans per PAA-Bio molecule) diluted in PBS+0.5% fish gelatin (FACS buffer) for 45 minutes on ice, followed by incubation with APC-Streptavidin (Southern Biotech) diluted 1:1000 in FACS buffer for 30 minutes on ice. Cells were washed in FACS buffer and cell fluorescence was measured by CytoFLEX flow cytometry (Beckman Coulter).

### Adoptive Cell Transfer

C57BL/6 were maintained in a Specific Pathogen-Free Facility of the Tel Aviv University. 0.25×10⁶ B16F10 cells in 100 μl were injected subcutaneous into the flank of 6 to 8-week-old male mice. Two treatment regimens were evaluated for CAR T administration. In the first regimen, on day 10 mice were irradiated at 2Gy, and on the following day mice were adoptively transferred with 7×10⁶ T cells in 500 μl PBS untransduced or RA0 CAR T cells via intravenous injections. In the second regimen, on day 3 mice were irradiated at 2Gy, and on the following day mice were adoptively transferred with 7×10⁶ T cells in 500 μl PBS untransduced or RA0 CAR T cells via intravenous injections. Tumor growth was monitored by a caliper every other day, and tumor volume calculated (tumor volume mm3=(length×width×depth)/2; n=5 per group).

### Statistical Analysis

Data was analyzed and graphed using Graphpad Prism V.8 (San Diego, CA, USA),as indicted in context. P value of 0.05 was considered statistically significant.

### Example 1. RA0 Antibody Target STn Specifically and Mediate Cancer Cells Killing

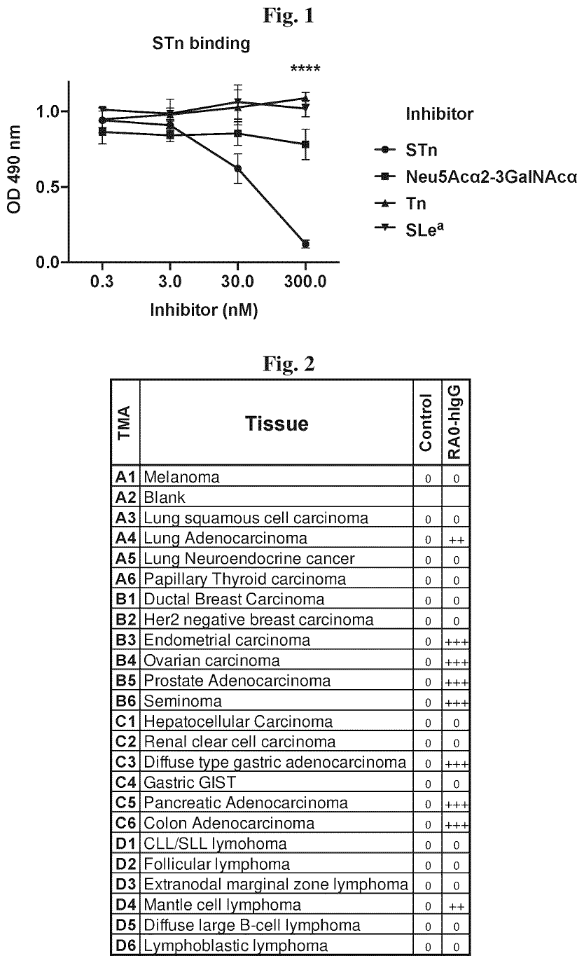

Variable domains of RA0 antibody were cloned into p3BNC plasmids containing the constant regions of human IgG Fc, and the full-length antibody was expressed in HEK293A cells. Competitive ELISA inhibition assay with this RA0-anti-STn-hIgG showed that only STn glycan antigen (STn conjugated to biotinylated polyacrylamide; STn-PAA-Bio) could inhibit binding to STn-coated plate, but not other closely-related glycans, including the non-sialylated Tn (GalNAcα-R), Neu5Acα2-3GalNAcα and sialyl-Lewis A (SLe^{a}) antigen (FIG. 1A). This is contrary to previous publications in which it was shown in a high-throughput glycan microarray analysis that RA0 binds specifically not only to STn (glycan Neu5Acα6GalNAcαO(CH2)2CH2NH2) but also closely-related structures (glycan IDs Neu5,9Ac2α6GalNAcαO(CH2)2CH2NH2; Neu5Acα6GalβO(CH2)2CH2NH2 and Neu5,9Ac2α6GalβO(CH2)2CH2NH2).

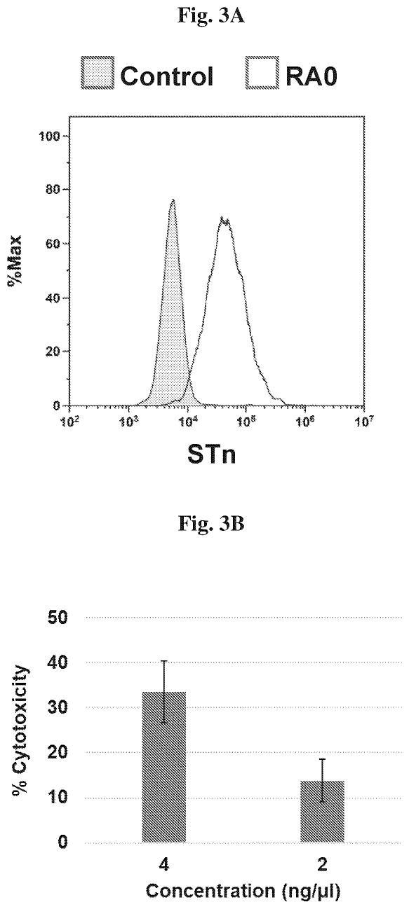

To evaluate the specificity of RA0 antibody against cancer tissues, human cancers tissue microarray (TMA) slides containing twenty-three different cancer tissues were stained by immunohistochemistry using biotinylated RA0 antibody (Bio-RA0-hIgG; prepared as described in the methods section). The TMA included samples from melanoma, lung squamous cell carcinoma, lung adenocarcinoma, lung neuroendocrine cancer, papillary thyroid carcinoma, ductal breast carcinoma, Her-2 negative breast carcinoma, endometrial carcinoma, ovarian carcinoma, prostate adenocarcinoma, seminoma, hepatocellular carcinoma, renal clear cell carcinoma, diffuse-type gastric adenocarcinoma, gastric GIST, pancreatic adenocarcinoma, colon adenocarcinoma, CLL/SLL lymphoma, follicular lymphoma, extranodal marginal zone lymphoma, mantle cell lymphoma, diffuse large B-cell lymphoma and lymphoblastic lymphoma. Of these tissues, endometrial carcinoma, ovarian carcinoma, prostate adenocarcinoma, seminoma, diffuse-type gastric adenocarcinoma, pancreatic and colon adenocarcinomas showed strong staining, lung adenocarcinoma and mantle cell lymphoma showed moderate staining, and the other tissues seemed to be negative for STn (FIG. 2). These results demonstrate that several adenocarcinomas showed a very high level of staining, providing a clear indication that these types of cancer extensively express STn and may be targeted for treatment using the CAR of the present invention that binds specifically to STn antigen. Furthermore, RA0 antibody could efficiently bind mouse melanoma B16F10 cells (FIG. 3A), and facilitate dose-dependent complement-dependent cytotoxicity (CDC) (FIG. 3B). Together, these results suggest that anti-RA0 antibodies and their derivatives can be used to specifically target cancer cells for therapy, potentially also in vivo.

### Example 2. CAR T Cells Bind STn and Induce In Vitro Cytotoxicity

To evaluate use of the antigen-binding domain of RA0 antibody in CAR-T therapy, the variable heavy and variable light chains of RA0 were synthesized, and linked them by a 3x(GGGGS) spacer to obtain a single-chain variable fragment (scFv). The scFv was incorporated into a CAR backbone containing a strep-tag connected through a 2x(GGGGS) spacer to the human CD28 transmembrane domain and intracellular co-stimulatory domain, followed by the FcγR ITAM intracellular signaling domain (FIG. 4) (referred also as RA0-CAR). The extracellular strep-tag allows to monitor CAR surface expression upon transduction. The CAR construct was cloned into the pMSGV1 retroviral vector, expressed in HEK 293T cells followed by generation of the PG-13 packaging cell line.

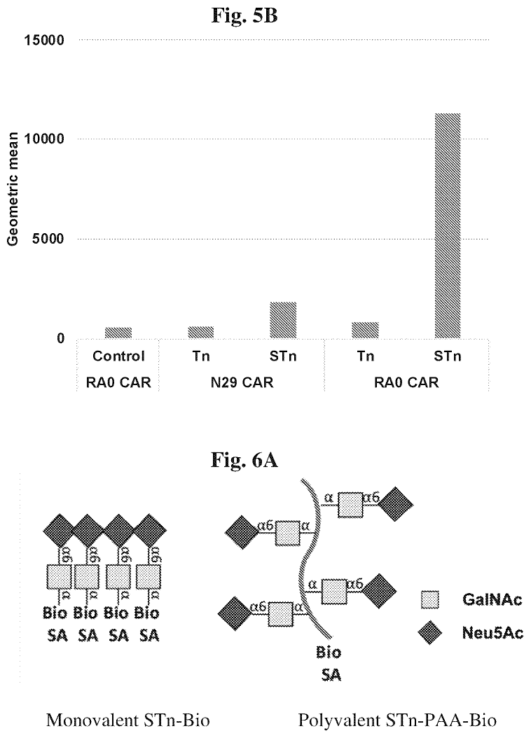

To demonstrate that the RA0-CAR construct maintained the high specificity against STn, the binding of RA0-CAR or irrelevant-CAR to the specific STn-PAA-Bio glycan target or the non-specific Tn-PAA-Bio glycan target that lacks the sialic acid moiety were examined. FACS analysis demonstrated that while the irrelevant-N29-CAR did not bind any glycan target, RA0-CAR could bind STn, but not to the non-sialylated Tn antigen (FIG. 5A and FIG. 5B), supporting the specificity of RA0-CAR against the STn target antigen.

The ability of the glycans to facilitate CAR stimulation through cytokines secretion was then evaluated. In this assay, T cells expressing RA0-CAR and untransduced (UT) T cells that do not express a surface chimeric receptor were compared. For this purpose, two different modes of glycans presentations, and their effects on cytokine release were examined: (1) condensed-rigid monovalent STn glycan with a terminal biotin at the non-reducing end (STn-Bio); and (2) dispersed and flexible polyvalent STn glycan conjugated at 6-8 copies onto a 30KDa polyacrylamide with a terminal biotin (STn-PAA-Bio), that better mimics the glycan presentation mode on the surface of cancer cells, as in mucins. While UT cells did not mediate any cytokine release, RA0-CAR T cells were stimulated only with the polyvalent STn-PAA-Bio glycans and facilitated secretion of IFN-γ (FIG. 6A) and TNF-α (FIG. 6B). These results support the efficacy of STn targeting by RA0-CAR T cells, particularly targeting STn glycans at high specificity that are expressed closely to their natural presentation mode. Likewise, RA0-CAR T cells were stimulated by STn-expressing-MEG-01 acute myeloid leukemia (AML) cells resulting in IFN-γ cytokine secretion (FIG. 6C).

### Example 3. RA0-CAR T Cells Inhibit Tumor Growth In Vivo in Mice

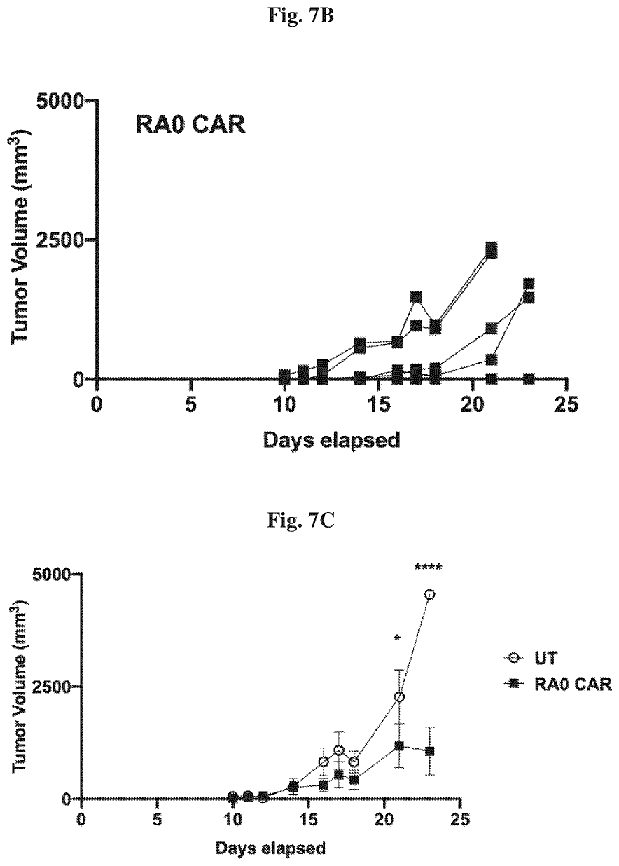

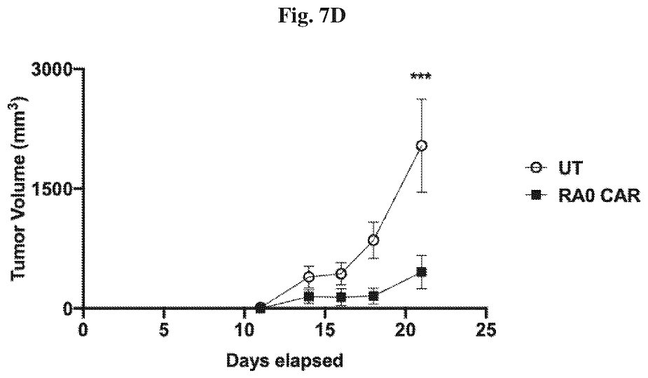

To assess the cytotoxicity of RA0-CAR T cells in vivo, B16F10 melanoma cells were subcutaneously injected into the flank of C57BL/6 mice. Once tumors were palpable, the mice were irradiated, and then treated on the next day by a single intravenous dose of about 7×10⁶ RA0-CAR T cells or of untransduced T (UT) cells as the control. Two treatment regimens were examined comparing late treatment (regimen 1: Day 10 irradiation, with next day treatment) or early treatment (regimen 2: Day 3 irradiation, with next day treatment). Results presented in FIG. 7 clearly show that a single administration of RA0-CAR T cells led to a significant inhibition of tumor growth compared to the control group (treated with untransduced T cells), in both treatment regimens (FIG. 7A-D). In treatment regimen 1, while the tumor volume in each mouse increased in the control UT group (FIG. 7A), in the RA0-CAR T cells treated group tumor growth was inhibited in each mouse (FIG. 7B; each line represents the kinetics of tumor growth in a single mouse). Furthermore, the mean tumor volume was significantly reduced in the RA0-CAR T cells treated group compared to the control untransduced T (UT) cells treated groups, in both treatment regimens: 1 (FIG. 7C), and 2 (FIG. 7D). Together, these results demonstrate the therapeutic potential of RA0-CAR against STn-expressing cancer cells. The evaluation of two different regimens demonstrated the high potency of the treatment, which might be suitable for different cancer stages, as CAR T administration in either day 4 or day 11 showed tumor growth inhibition. Given that about 80% of human carcinoma express STn, this could provide a robust novel immunotherapy approach.

### Example 4. RA0-CAR T Cells Inhibit Tumor Growth In Vivo in Mice

To assess the cytotoxicity of RA0-CAR T cells in vivo, cells being a model of each one of the following types of cancer: lung, breast Her-2 negative, breast Her-2 positive, ovarian, pancreatic adenocarcinoma, colorectal, stomach, liver, oropharyngeal cancer, head and neck and gallbladder cancer, pharynx squamous cell carcinoma, leukemia, lymphoma and myeloma are subcutaneously injected into the flank of C57BL/6 mice. Once tumors are palpable, the mice are irradiated, and then treated on the next day by a single intravenous dose of RA0-CAR T cells or of untransduced T (UT) cells as the control. Two treatment regimens are examined comparing late treatment (regimen 1: Day 10 irradiation, with next day treatment) or early treatment (regimen 2: Day 3 irradiation, with next day treatment). Results show that a single administration of RA0-CAR T cells lead to a significant inhibition of tumor growth compared to the control group (treated with untransduced T cells), in both treatment regimens. These results demonstrate the therapeutic potential of RA0-CAR against STn-expressing cancer cells. The evaluation of two different regimens demonstrate the high potency of the treatment, which might be suitable for different cancer stages, as CAR T administration in either day 4 or day 11 showed tumor growth inhibition. Given that about 80% of human carcinoma express STn, this could provide a robust novel immunotherapy approach.

Although the present invention has been described herein by way of preferred embodiments thereof, it can be modified, without departing from the spirit and nature of the subject invention as defined in the appended claims

1-41. (canceled)

42. A chimeric antigen receptor (CAR) comprising an antigen-binding domain that binds specifically to Sialyl Tn glycan (STn), wherein the antigen-binding domain comprises three complementarity determining regions (CDRs) of a heavy-chain variable domain (VH) having an amino acid sequence as set forth in SEQ ID NO: 1 and three CDRs of a light-chain variable domain (VL) having an amino acid sequence as set forth in SEQ ID NO: 2.

43. The CAR according to claim 42, wherein the VH-CDR1 comprises amino acid sequence SEQ ID NO: 3; VH-CDR2 comprises amino acid sequence SEQ ID NO: 4; VH-CDR3 comprises amino acid sequence SEQ ID NO: 5; VL-CDR1 comprises amino acid sequence SEQ ID NO: 6; VL-CDR2 comprises amino acid sequence SEQ ID NO: 7; and VL-CDR3 comprises amino acid sequence SEQ ID NO: 8.

44. The CAR according to claim 42, wherein the VH domain comprises amino acid sequence SEQ ID NO: 1 or a sequence having at least 95% sequence identity to SEQ IDN NO: 1 and comprising 3 CDRs: VH-CDR1, VH-CDR3 and VH-CDR3 comprising the amino acid sequences SEQ ID NO: 3, 4 and 5, respectively and wherein the VL domain comprises amino acid sequence SEQ ID NO: 2 or a sequence having at least 95% sequence identity to SEQ IDN NO: 2 and comprising 3 CDRs: VL-CDR1, VL-CDR3 and VL-CDR3 comprising the amino acid sequences SEQ ID NO: 6, 7 and 8, respectively.

45. The CAR according to claim 42, wherein the VH and the VL domains are linked by a spacer to form a single-chain variable fragment (scFv).

46. The CAR according to claim 45, wherein the CAR is characterized by at least one of:
(i) the spacer comprises an amino acid sequence comprising from 2 to 6 repetitions of the amino acid sequence set forth in SEQ ID NO: 9;
(ii) the scFv comprises the amino acid sequence SEQ ID NO: 11 or an amino acid sequence having at least 95% to SEQ ID NO: 11 and comprising 6 CDRs: VH-CDR1, VH-CDR3 and VH-CDR3 comprising the amino acid sequence SEQ ID NO: 3, 4 and 5, respectively and VL-CDR1, VL-CDR3 and VL-CDR3 comprising the amino acid sequences SEQ ID NO: 6, 7 and 8; and
(iii) wherein the CAR comprises a transmembrane domain (TM domain), a costimulatory domain, and an activation domain.

47. The CAR according to claim 46, characterized by at least one of:
(i) the TM domain is a TM domain of a receptor selected from CD28 and CD8;
(ii) the costimulatory domain is selected from a costimulatory domain of a protein selected from CD28, 4-1BB, OX40, iCOS, CD27, CD80, and CD70;
(iii) the TM domain and the costimulatory domain are both derived from CD28;
(iv) the activation domain is selected from FcRγ and CD3-3 activation domains
(iv) the TM domain and the costimulatory domain have amino acid sequence SEQ ID NO: 12;
(vi) the antigen-binding domain is linked to the TM domain via a spacer; and
(v) the CAR further comprises a leading peptide.

48. The CAR according to claim 47, characterized by at least one of:
(i) the spacer comprises an amino acid sequence comprising from 1 to 4 repetitions of amino acid sequence SEQ ID NO: 9;
(ii) the activation domain is FcRγ having amino acid sequence SEQ ID NO: 13; and
(iii) the leading peptide has amino acid sequence SEQ ID NO: 14.

49. The CAR according to claim 42, comprising an amino acid sequence selected from SEQ ID NO: 15 16, and a sequence having at least 95% sequence identity to sequence 15 or 16 and comprising 6 CDRs: VH-CDR1, VH-CDR3 and VH-CDR3 comprising the amino acid sequence SEQ ID NO: 3, 4 and 5, respectively and VL-CDR1, VL-CDR3 and VL-CDR3 comprising the amino acid sequences SEQ ID NO: 6, 7 and 8.

50. A cell comprising the CAR according to claim 42.

51. The cell according to claim 50, wherein the cell is selected from a T cell and a natural killer (NK) cell.

52. A nucleic acid molecule encoding the CAR according to claim 42.

53. The nucleic acid molecule according to claim 52, characterized by at least one of:
(i) the nucleic acid molecule encodes an amino acid sequence selected from SEQ ID NO: 1, SEQ ID NO: 2, both SEQ ID NOs: 1 and 2, a amino acid sequence having at least 95% sequence identity to SEQ IDN NO: 1 and comprising 3 CDRs: VH-CDR1, VH-CDR3 and VH-CDR3 comprising the amino acid sequences SEQ ID NO: 3, 4 and 5, respectively, and an amino acid sequence having at least 95% sequence identity to SEQ IDN NO: 2 and comprising 3 CDRs: VL-CDR1, VL-CDR3 and VL-CDR3 comprising the amino acid sequences SEQ ID NO: 6, 7 and 8, respectively
(ii) comprising a nucleic acid sequence selected from SEQ ID NO: 17, SEQ ID NO: 18, a variant of SEQ ID NO: 17 or 18; and
(iii) the nucleic acid molecule encodes the amino acid sequence SEQ ID NO: 11 or an amino acid sequence having at least 95% to SEQ ID NO: 11 and comprising 6 CDRs: VH-CDR1, VH-CDR3 and VH-CDR3 comprising the amino acid sequence SEQ ID NO: 3, 4 and 5, respectively and VL-CDR1, VL-CDR3 and VL-CDR3 comprising the amino acid sequences SEQ ID NO: 6, 7 and 8;
(iv) comprising a nucleic acid sequence selected from SEQ ID NO: 20.

54. The nucleic acid molecule according to claim 53, characterized by one of the following:
(i) the nucleic acid molecule further encodes an amino acid sequence selected from SEQ ID NOs: 12, 13, 14;
(ii) the nucleic acid molecule further comprises a nucleic acid sequence selected from SEQ ID NO: 22, 23, 24
(iii) the nucleic acid encodes amino acid sequence selected from SEQ ID NO: 15, SEQ ID NO: 16 and a sequence having at least 95% sequence identity to sequence 15 or 16 and comprising 6 CDRs: VH-CDR1, VH-CDR3 and VH-CDR3 comprising the amino acid sequence SEQ ID NO: 3, 4 and 5, respectively and VL-CDR1, VL-CDR3 and VL-CDR3 comprising the amino acid sequences SEQ ID NO: 6, 7 and;
(iv) comprising a nucleic acid sequence selected from SEQ ID NO: 25, SEQ ID NO: 26.

55. A nucleic acid construct comprising the nucleic acid molecule according to claim 52, operably linked to a promoter.

56. A vector comprising the nucleic acid molecule according to claim 52 or the nucleic acid construct comprising the nucleic acid molecule.

57. A cell comprising the nucleic acid molecule according to claim 52, the nucleic acid construct or the vector comprising same.

58. The cell according to claim 57, wherein the cell is characterized by at least one of:
(i) The cell expresses or is capable of expressing the CAR encoded by the nucleic acid, construct or vector, wherein the CAR comprising an antigen-binding domain that binds specifically to Sialyl Tn glycan (STn), wherein the antigen-binding domain comprises three complementarity determining regions (CDRs) of a heavy-chain variable domain (VH) having an amino acid sequence as set forth in SEQ ID NO: 1 and three CDRs of a light-chain variable domain (VL) having an amino acid sequence as set forth in SEQ ID NO: 2; and
(ii) the cell is selected from a T cell and a natural killer (NK) cell.

59. A pharmaceutical composition comprising a plurality of cells according claim 57.

60. A pharmaceutical composition comprising a plurality of cells according claim 50.

61. A method for treating cancer in a subject in need thereof comprising administering a therapeutically effective amount of cells comprising the CAR according to claim 42 or a nucleic acid molecule encoding same.


SEQUENCE LISTING (26 sequences)

SEQ ID NO: 1 - protein, 113 aa, Artificial Sequence
  Synthesized sequence
QVQLQESGPGLVAPSQSLSITCTVSGFSLISYGVSWVRQPPGKGLEWLGVIWGDGSTNYHSTLISRLSINKDNSKSQVFLKLNSLQTDDTATYYCVGPRFAYWGQGTLVTVSA

SEQ ID NO: 2 - protein, 106 aa, Artificial Sequence
  Synthesized sequence
QIVLTQSPAIMSASPGEKVTMTCSASSSVSYMHWYQQKSGTSPKRWIYDTSKLTSGVPARFSGSGSGTSYSLTISSMEAEDAATYYCQQWSSNTLTFGAGTKLELK

SEQ ID NO: 3 - protein, 5 aa, Artificial Sequence
  Synthesized sequence
SYGVS

SEQ ID NO: 4 - protein, 16 aa, Artificial Sequence
  Synthesized sequence
VIWGDGSTNYHSTLIS

SEQ ID NO: 5 - protein, 5 aa, Artificial Sequence
  Synthesized sequence
PRFAY

SEQ ID NO: 6 - protein, 10 aa, Artificial Sequence
  Synthesized sequence
SASSSVSYMH

SEQ ID NO: 7 - protein, 7 aa, Artificial Sequence
  Synthesized sequence
DTSKLTS

SEQ ID NO: 8 - protein, 9 aa, Artificial Sequence
  Synthesized sequence
QQWSSNTLT

SEQ ID NO: 9 - protein, 5 aa, Artificial Sequence
  Synthesized sequence
GGGGS

SEQ ID NO: 10 - protein, 15 aa, Artificial Sequence
  Synthesized sequence
GGGGSGGGGSGGGGS

SEQ ID NO: 11 - protein, 234 aa, Artificial Sequence
  Synthesized sequence
QVQLQESGPGLVAPSQSLSITCTVSGFSLISYGVSWVRQPPGKGLEWLGVIWGDGSTNYHSTLISRLSINKDNSKSQVFLKLNSLQTDDTATYYCVGPRFAYWGQGTLVTVSAGGGGSGGGGSGGGGSQIVLTQSPAIMSASPGEKVTMTCSASSSVSYMHWYQQKSGTSPKRWIYDTSKLTSGVPARFSGSGSGTSYSLTISSMEAEDAATYYCQQWSSNTLTFGAGTKLELK

SEQ ID NO: 12 - protein, 110 aa, Artificial Sequence
  Synthesized sequence
AAAIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRS

SEQ ID NO: 13 - protein, 38 aa, Artificial Sequence
  Synthesized sequence
QVRKAAITSYEKSDGVYTGLSTRNQETYETLKHEKPPQ

SEQ ID NO: 14 - protein, 22 aa, Artificial Sequence
  Synthesized sequence
MDFQVQIFSFLLISASVIMSRG

SEQ ID NO: 15 - protein, 422 aa, Artificial Sequence
  Synthesized sequence
MDFQVQIFSFLLISASVIMSRGQVQLQESGPGLVAPSQSLSITCTVSGFSLISYGVSWVRQPPGKGLEWLGVIWGDGSTNYHSTLISRLSINKDNSKSQVFLKLNSLQTDDTATYYCVGPRFAYWGQGTLVTVSAGGGGSGGGGSGGGGSQIVLTQSPAIMSASPGEKVTMTCSASSSVSYMHWYQQKSGTSPKRWIYDTSKLTSGVPARFSGSGSGTSYSLTISSMEAEDAATYYCQQWSSNTLTFGAGTKLELKWSHPQFEKGGGGSGGGGSAAAIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSQVRKAAITSYEKSDGVYTGLSTRNQETYETLKHEKPPQ

SEQ ID NO: 16 - protein, 414 aa, Artificial Sequence
  Synthesized sequence
MDFQVQIFSFLLISASVIMSRGQVQLQESGPGLVAPSQSLSITCTVSGFSLISYGVSWVRQPPGKGLEWLGVIWGDGSTNYHSTLISRLSINKDNSKSQVFLKLNSLQTDDTATYYCVGPRFAYWGQGTLVTVSAGGGGSGGGGSGGGGSQIVLTQSPAIMSASPGEKVTMTCSASSSVSYMHWYQQKSGTSPKRWIYDTSKLTSGVPARFSGSGSGTSYSLTISSMEAEDAATYYCQQWSSNTLTFGAGTKLELKGGGGSGGGGSAAAIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSQVRKAAITSYEKSDGVYTGLSTRNQETYETLKHEKPPQ

SEQ ID NO: 17 - DNA, 339 bp, Artificial Sequence
  Synthesized sequence
caggtgcagctgcaggagtctggacctggcctggtggcgccctcacagagcctgtccatcacatgcactgtctcagggttctcattaatcagctatggtgtaagctgggttcgccagcctccaggaaagggtctggagtggctgggagtaatatggggtgacgggagcacaaattatcattcaactctcatatccagactgagcatcaacaaggataactccaagagccaagttttcttaaaactgaacagtctgcaaactgatgacacagccacgtactactgtgtcggaccccggtttgcttactggggccaagggactctggtcactgtctctgca

SEQ ID NO: 18 - DNA, 318 bp, Artificial Sequence
  Synthesized sequence
caaattgttctcacccagtctccagcaatcatgtctgcatctccaggggagaaggtcaccatgacctgcagtgccagctcaagtgtaagttacatgcactggtaccagcagaagtcaggcacctcccccaaaagatggatttatgacacatccaaactgacttctggagtccctgctcgcttcagtggcagtgggtctgggacctcttactctctcacaatcagcagcatggaggctgaagatgctgccacttattactgccagcagtggagtagtaatacgctcacgttcggtgctgggaccaagctggagctgaaa

SEQ ID NO: 19 - DNA, 45 bp, Artificial Sequence
  Synthesized sequence
ggaggtggcggtagcggaggcggcggttctggaggtggcgggagc

SEQ ID NO: 20 - DNA, 702 bp, Artificial Sequence
  Synthesized sequence
caggtgcagctgcaggagtctggacctggcctggtggcgccctcacagagcctgtccatcacatgcactgtctcagggttctcattaatcagctatggtgtaagctgggttcgccagcctccaggaaagggtctggagtggctgggagtaatatggggtgacgggagcacaaattatcattcaactctcatatccagactgagcatcaacaaggataactccaagagccaagttttcttaaaactgaacagtctgcaaactgatgacacagccacgtactactgtgtcggaccccggtttgcttactggggccaagggactctggtcactgtctctgcaggaggtggcggtagcggaggcggcggttctggaggtggcgggagccaaattgttctcacccagtctccagcaatcatgtctgcatctccaggggagaaggtcaccatgacctgcagtgccagctcaagtgtaagttacatgcactggtaccagcagaagtcaggcacctcccccaaaagatggatttatgacacatccaaactgacttctggagtccctgctcgcttcagtggcagtgggtctgggacctcttactctctcacaatcagcagcatggaggctgaagatgctgccacttattactgccagcagtggagtagtaatacgctcacgttcggtgctgggaccaagctggagctgaaa

SEQ ID NO: 21 - DNA, 45 bp, Artificial Sequence; identical to SEQ ID NO: 19
  Synthesized sequence

SEQ ID NO: 22 - DNA, 330 bp, Artificial Sequence
  Synthesized sequence
gcggccgcaattgaagttatgtatcctcctccttacctagacaatgagaagagcaatggaaccattatccatgtgaaagggaaacacctttgtccaagtcccctatttcccggaccttctaagcccttttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatagatct

SEQ ID NO: 23 - DNA, 117 bp, Artificial Sequence
  Synthesized sequence
caagtgcgaaaggcagctataaccagctatgagaaatcagatggtgtttacacgggcctgagcaccaggaaccaggagacttacgagactctgaagcatgagaaaccaccacagtag

SEQ ID NO: 24 - DNA, 66 bp, Artificial Sequence
  Synthesized sequence
atggattttcaggtgcagattttcagcttcctgctaatcagtgcctcagtcataatgtccagagga

SEQ ID NO: 25 - DNA, 1269 bp, Artificial Sequence
  Synthesized sequence
atggattttcaggtgcagattttcagcttcctgctaatcagtgcctcagtcataatgtccagaggacaggtgcagctgcaggagtctggacctggcctggtggcgccctcacagagcctgtccatcacatgcactgtctcagggttctcattaatcagctatggtgtaagctgggttcgccagcctccaggaaagggtctggagtggctgggagtaatatggggtgacgggagcacaaattatcattcaactctcatatccagactgagcatcaacaaggataactccaagagccaagttttcttaaaactgaacagtctgcaaactgatgacacagccacgtactactgtgtcggaccccggtttgcttactggggccaagggactctggtcactgtctctgcaggaggtggcggtagcggaggcggcggttctggaggtggcgggagccaaattgttctcacccagtctccagcaatcatgtctgcatctccaggggagaaggtcaccatgacctgcagtgccagctcaagtgtaagttacatgcactggtaccagcagaagtcaggcacctcccccaaaagatggatttatgacacatccaaactgacttctggagtccctgctcgcttcagtggcagtgggtctgggacctcttactctctcacaatcagcagcatggaggctgaagatgctgccacttattactgccagcagtggagtagtaatacgctcacgttcggtgctgggaccaagctggagctgaaatggagccacccgcagttcgagaaaggtggaggtggatccggtggaggtggatccgcggccgcaattgaagttatgtatcctcctccttacctagacaatgagaagagcaatggaaccattatccatgtgaaagggaaacacctttgtccaagtcccctatttcccggaccttctaagcccttttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatagatctcaagtgcgaaaggcagctataaccagctatgagaaatcagatggtgtttacacgggcctgagcaccaggaaccaggagacttacgagactctgaagcatgagaaaccaccacagtag

SEQ ID NO: 26 - DNA, 1245 bp, Artificial Sequence
  Synthesized sequence
atggattttcaggtgcagattttcagcttcctgctaatcagtgcctcagtcataatgtccagaggacaggtgcagctgcaggagtctggacctggcctggtggcgccctcacagagcctgtccatcacatgcactgtctcagggttctcattaatcagctatggtgtaagctgggttcgccagcctccaggaaagggtctggagtggctgggagtaatatggggtgacgggagcacaaattatcattcaactctcatatccagactgagcatcaacaaggataactccaagagccaagttttcttaaaactgaacagtctgcaaactgatgacacagccacgtactactgtgtcggaccccggtttgcttactggggccaagggactctggtcactgtctctgcaggaggtggcggtagcggaggcggcggttctggaggtggcgggagccaaattgttctcacccagtctccagcaatcatgtctgcatctccaggggagaaggtcaccatgacctgcagtgccagctcaagtgtaagttacatgcactggtaccagcagaagtcaggcacctcccccaaaagatggatttatgacacatccaaactgacttctggagtccctgctcgcttcagtggcagtgggtctgggacctcttactctctcacaatcagcagcatggaggctgaagatgctgccacttattactgccagcagtggagtagtaatacgctcacgttcggtgctgggaccaagctggagctgaaaggtggaggtggatccggtggaggtggatccgcggccgcaattgaagttatgtatcctcctccttacctagacaatgagaagagcaatggaaccattatccatgtgaaagggaaacacctttgtccaagtcccctatttcccggaccttctaagcccttttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatagatctcaagtgcgaaaggcagctataaccagctatgagaaatcagatggtgtttacacgggcctgagcaccaggaaccaggagacttacgagactctgaagcatgagaaaccaccacagtag